(19) United States
(12) Patent Application Publication
(10) Pub. No.: US 2025/0305073 A1
(43) Pub. Date: Oct. 2, 2025

(54) SYSTEMS AND METHODS FOR VIRUS DETECTION

(71) Applicant: University of Connecticut, Farmington, CT (US)

(72) Inventors: Changchun Liu, Farmington, CT (US); Ziyue Li, Farmington, CT (US)

(21) Appl. No.: 19/089,558

(22) Filed: Mar. 25, 2025

Related U.S. Application Data
(60) Provisional application No. 63/569,749, filed on Mar. 26, 2024.

(51) Int. Cl. C12Q 1/70; B01L 3/00; B01L 7/00; C12Q 1/6844

(52) U.S. Cl. CPC C12Q 1/701; B01L 3/502761; B01L 7/52; C12Q 1/6844; C12Q 1/703; B01L 2200/0668; B01L 2200/16; B01L 2200/18; B01L 2300/18

(57) ABSTRACT

The present disclosure relates to composition, systems and methods for a wide variety of sample analysis applications, including nucleic acid amplification assays. The system and method includes an integrated, lab-in-a magnetofluidic tube (LIAMT) platform that integrates virus lysis, nucleic acid extraction, isothermal amplification, and CRISPR detection within a single system for molecular detection of viruses.

### RELATED APPLICATIONS

This application claims priority to provisional patent application 63/569,749, filed Mar. 26, 2024, which is herein incorporated by reference in its entirety.

### GOVERNMENT SUPPORT

This invention was made with government support under EB023607, and AI154642 awarded by the National Institutes of Health. The government has certain rights in the invention.

### FIELD OF THE DISCLOSURE

Described herein are systems and methods for molecular detection of virus, especially, the system and methods including a simple, fully integrated, lab-in-a-magnetofluidic tube (LIAMT) platform for “sample-to-result” molecular detection of virus.

### BACKGROUND

Simple, rapid, and sensitive nucleic acid-based molecular detection of virus is essential for monitoring and controlling the spread of infectious diseases, including COVID-19 caused by the severe acute respiratory syndrome coronavirus 2 (SARS-CoV-2) and acquired immune deficiency syndrome caused by human immunodeficiency virus (HIV). To achieve highly sensitive and specific molecular detection in clinical samples, nucleic acid-based diagnostics typically consist of three major steps: i) nucleic acid extraction, ii) enzymatic amplification, and iii) signal detection. Polymerase chain reaction (PCR)/reverse transcription PCR (RT-PCR) is considered the “gold standard” for nucleic acid amplification testing due to its high sensitivity and specificity. However, current PCR/RT-PCR assays require extensive nucleic acid sample preparation and expensive instrumentation for precise thermal cycling, both of which limit the feasibility for point-of-care diagnostic applications. Therefore, there is an unmet need to develop a simple, integrated, “sample-to-result” molecular diagnostic tool that can be used at the point of care, particularly in resource-limited settings.

### SUMMARY

Described herein are systems and methods for detection of pathogens, especially for a wide variety of sample analysis applications, including nucleic acid amplification assays.

Aspects of the present disclosure provide a simple, affordable, portable, and sensitive LIAMT platform for the “sample-to-result” nucleic acid detection at the point-of-care. The compositions, systems, and methods find use, for example, for rapid detection of infectious disease at the point-of-care, especially in resource-limited settings.

In an aspect, disclosed is a system including an integrated, lab-in-a magnetofluidic tube (LIAMT) platform/device for a molecular detection of virus as shown and described herein.

In an aspect, disclosed is a method for a molecular detection of virus using the disclosed lab-in-a magnetofluidic tube (LIAMT) system as shown and described herein.

For example, in some embodiments, provided herein is a device, comprising: a reaction vessel comprising a plurality of distinct assay zones comprising a) a lysis zone comprising a lysis/binding buffer and magnetic beads; b) one or more (e.g., 1, 2, 3, or more) washing zones comprising a washing buffer; c) an amplification zone comprising amplification reagents; and d) a detection zone comprising nucleic acid detection reagents.

The present disclosure is not limited to particular configurations of the reaction vessel. In some embodiment, the assay zones are vertically oriented columns in the reaction vessel. In some embodiments, the assay zones are separated by a separator. In some embodiments, the top of the reaction vessel is covered with mineral oil in fluid contact with the top surface of each of the assay zones. In certain aspects, the amplification zone and the detection zone are in the same assay zone and are separated by temperature-sensitive wax (e.g., n-Eicosane wax). In some embodiments, the device further comprises a blood sample zone comprising a plasma separation membrane. In some embodiments, the reaction chamber is a reaction tube. In some embodiments, the device is disposable.

Any number of suitable buffers and assay reagents may be utilized in the devices described herein. For example, in some embodiments, the wash buffer is MgOAc (e.g., 50 mM MgOAc) with 1% Triton-X-100. In some embodiments, the amplification reagents are reagents for isothermal amplification (e.g., recombinase polymerase amplification (RPA) or reverse transcriptase RPA (RT-RPA)) of a target nucleic acid (e.g., nucleic acid primers, nucleotides, buffer, and/or polymerases).

Embodiments of the disclosure utilize nucleic acid detection reagents are reagents for performing a CRISPR assay (e.g., a guide RNA, a RNA-guided endonuclease, a reporter probe, and/or a buffer). In some embodiments, the reporter is a fluorescence reporter (e.g., dye/quencher-labeled single-stranded DNA fluorescence probe). In some embodiments, the guide RNA is a combination of a tracrRNA and a crRNA or an sgRNA, In some embodiments, the guided endonuclease is Cas12A.

Further embodiments provide a system, comprising a device as described herein and a sample processor comprising a magnetofluidic separation well for magnetofluidic transferring and operation, and an incubation/detection well. In some embodiments, the magnetofluidic well comprises a magnet (e.g., a neodymium magnet). In some embodiments, the incubation/detection well comprises a heater (e.g., thin-film heater) and a thermocouple wire. In some embodiments, the sample processor comprises a single well for both magnetofluidic separation and incubation/detection. In some embodiments, the system further comprises one or more of a microprocessor, a plurality of first stepper motors configured to transfer magnetic beads within assay zones of the device and a plurality of second stepper motors configured to move the device between the magnetofluidic well and the incubation/detection well, and a camera. In some embodiments, the microprocessor is configured to real-time monitor the fluorescence signals, analyze the fluorescence images and quantify the nucleic acid target. In some embodiments, the system is fully automated.

Additional embodiments of the disclosure provide a method for detection of a target nucleic acid in a sample, comprising: contacting the sample with a system described herein; and detecting the presence and/or level of the nucleic acid in the sample using the system. In some embodiments, the method comprises transferring the magnetic beads to the washing buffer zones for nucleic acid purification by manually rotating and lifting the device or by the stepper motor. In some embodiments, the nucleic acid (e.g., DNA or RNA) is a pathogen nucleic acid (e.g., a viral nucleic acid). In some embodiments, the virus is SARS-CoV-2 or HIV. In certain embodiments, the sample is from a subject.

These and other aspects and embodiments of the disclosure are described in more detail below.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIGS. 1A-1C. Overview of the LIAMT assay. FIG. 1A) Clinical assay workflow of the LIAMT platform. FIG. 1B) Schematic illustration of the LIAMT device. The device contains five independent zones with pre-stored lysis/binding buffer, washing buffer, and RT-RPA/wax/CRISPR reaction solution, respectively. FIG. 1C) Schematic shows the processing of clinical sample detection by the magnetofluidic separation operation in the LIAMT platform. This includes three main steps: 1) adding the clinical sample into the LIAMT device and mixing with lysis/binding buffer containing magnetic beads, 2) washing the magnetic beads bound with nucleic acids in washing buffer zones to purify nucleic acids, and 3) transferring the magnetic beads with nucleic acids into the RT-RPA/wax/CRISPR zone for isothermal amplification and CRISPR detection.

FIGS. 2A-2G. Optimization of magnetofluidic-based nucleic acid detection. FIG. 2A & FIG. 2B) Comparison and optimization of five lysis/binding buffer solutions (ZYMO, MES, MgOAc, Tris-HCl, and Tris-HCl+IPA) and four washing buffer solutions (H₂O, 70% ethanol, 10 mM MgOAc with 1% Triton X-100, and 20% PEG solution) by RT-RPA/CRISPR and RT-PCR, respectively. FIG. 2C) Six different concentrations (1, 5, 10, 50, 100, and 500 mM) of MgOAc solution with 1% Triton X-100 were prepared and tested as washing buffer in the LIAMT device with various SARS-CoV-2 concentrations (3,600, 360, and 36 copies/μL). FIG. 2D) Effect of the magnetic bead volume (0.5, 1, 2, 4, 8, and 16 μL) on the magnetofluidic-based nucleic acid extraction. FIG. 2E) Effect of the washing buffer volume ranging from 120 to 420 μL on the magnetofluidic-based nucleic acid extraction. FIG. 2F) Effect of the number of washing steps on the magnetofluidic-based nucleic acid detection. FIG. 2G) Comparison and optimization of the incubation time for the RT-RPA reaction and CRISPR reaction, respectively.

FIGS. 3A-3E. LIAMT platform and its operation. FIG. 3A) Photograph of the LIAMT device and its 3D-printed separator. Inset is the top view of the separator. FIG. 3B) Magnetofluidic-based nucleic acid extraction steps in the LIAMT device. 1) Clinical sample is added into the device and mixed with the lysis/binding buffer containing magnetic beads. 2) The LIAMT device is rotated clockwise to the first washing buffer zone (as indicated by “2” on the cap) and then lifted for the first washing step. 3 & 4) The magnetic beads are washed two more times by repeating the magnetofluidic separation operation. 5) The magnetic beads bound with nucleic acids are transferred into the RPA/wax/CRISPR reaction zone. FIG. 3C) Schematic illustration of the palm-sized processor, which contains a magnetofluidic separation well for magnetofluidic-based nucleic acid extraction, and an incubation/detection well for isothermal amplification and CRISPR detection. FIG. 3D) Comparison of nucleic acid sample preparation time between the traditional spin column-based method and the magnetofluidic method of the LIAMT device. FIG. 3E) Photograph of the palm-sized processor. Endpoint fluorescence images of 1) SARS-CoV-2-positive and 2) SARS-CoV-2-negative samples.

FIGS. 4A-4D. Virus detection in spiked samples by using the LIAMT platform. FIG. 4A) Normalized fluorescence intensity of the device for SARS-CoV-2 detection. NC, negative control. n.s., not significant with p>0.05. ****, P<0.00001. FIG. 4B) Real-time fluorescence detection of the CRISPR reaction after separate RPA preamplification of SARS-CoV-2 in the spiked samples. n=3. FIG. 4C) Normalized fluorescence intensity for HIV detection in the LIAMT device. FIG. 4D) Real-time fluorescence detection of RPA/CRISPR reaction of HIV in the plasma samples. n=3.

FIGS. 5A-5F. Clinical validation of the LIAMT platform to detect SARS-CoV-2 and HIV in clinical samples. FIG. 5A) Normalized fluorescence intensity of the LIAMT device for SARS-CoV-2 detection in 32 clinical swab samples. FIG. 5B) ROC curve of the LIAMT results for SARS-CoV-2 detection. ROC curve analysis of the device compared to the standard RT-PCR method. FIG. 5C) Statistical analysis of the LIAMT results for SARS-CoV-2 detection.

FIG. 5D) Normalized fluorescence intensity of the LIAMT device for HIV detection in 41 clinical plasma samples. FIG. 5E) ROC curve of the LIAMT results for HIV detection. FIG. 5F) Statistical analysis of the LIAMT results for HIV detection.

FIG. 6. Exploded view of the palm-sized processor, which includes: \textcircled{1} an emission filter, \textcircled{2} an excitation filter, \textcircled{3} a control circuit board, \textcircled{4} a flexible heater combined with a thermocouple wire, \textcircled{5} an LED light, and \textcircled{6} a neodymium magnet.

FIGS. 7A-7C. Digital RT-PCR quantification of SARS-CoV-2 by QuantStudio 3D digital PCR chips. FIG. 7A) Fluorescence images of QuantStudio 3D digital PCR chips for detection of various SARS-CoV-2 concentrations. FIG. 7B) Absolute quantification of SARS-CoV-2 viral load by digital PCR in a dilution series. FIG. 7C) Linear regression of SARS-CoV-2 copy numbers and genome equivalents (GE) by digital PCR. Statistical significance was determined by performing an analysis of variance (ANOVA) test. Different letters were considered to be significantly different.

FIGS. 8A-8C. Digital RT-PCR quantification of HIV by QuantStudio 3D digital PCR chips. FIG. 8A) Fluorescence images of QuantStudio 3D digital PCR chips for detection of various HIV serial dilutions. FIG. 8B) Absolute quantification of SARS-CoV-2 viral load by digital PCR in HIV serial dilutions. FIG. 8C) Linear regression of HIV copy numbers with serial dilutions by digital PCR. Statistical significance was determined by performing an ANOVA test. Different letters were considered to be significantly different.

FIG. 9. Cq values of SARS-CoV-2 detection in 32 clinical samples by real-time RT-PCR.

FIG. 10. Endpoint fluorescence images of the LIAMT device for the detection of different concentrations of SARS-CoV-2 in the spiked samples.

FIG. 11. Endpoint fluorescence detection of CRISPR reactions after separate RT-RPA preamplification for SARS-CoV-2 detection in 32 clinical samples.

FIG. 12. Cq values of HIV detection in 41 clinical samples by real-time RT-PCR.

FIG. 13. Endpoint fluorescence images of the LIAMT device for the detection of different concentrations of SARS-CoV-2 in the spiked samples.

FIG. 14. Endpoint fluorescence detection of CRISPR reactions after separate RT-RPA preamplification for HIV detection in 41 clinical samples.

FIG. 15. Schematic illustration of the LIAMT device incorporated with Vivid plasma separation membrane. Shown are six independent zones: 1) whole blood zone, 2) lysis/binding buffer zone, 3-5) washing buffer, and 6) RT-RPA/CRISPR reaction zone, respectively.

FIGS. 16A-16B. Raspberry Pi processor. FIG. 16A) Schematic illustration of the portable Raspberry Pi processor for automatic magnetofluidic operation and detection of the LIAMT device. FIG. 16B) Photograph of the portable Raspberry Pi processor for the LIAMT device.

### DETAILED DESCRIPTION

Disclosed herein are systems or platforms or devices and methods including a simple, fully-integrated, lab-in-a magnetofluidic tube (LIAMT) platform for “sample-to-result”, molecular detection of target nucleic acids (e.g., viral nucleic acids) in a subject such as mammals, in particular humans.

In recent decades, researchers have explored different strategies to develop simple, rapid, and affordable point-of-care diagnostic technologies for infectious disease detection. Some studies have reported nucleic acid extraction-free molecular diagnostic assays by combining simple heat-treated sample preparation with nucleic acid amplification tests such as PCR and loop-mediated isothermal amplification. However, detecting low-abundance target molecules in clinical samples without nucleic acid extraction and purification remains challenging. To meet stringent sensitivity requirements in clinical testing, solid phase extraction of nucleic acids has been integrated into microfluidic chips to develop fully integrated molecular diagnostic platforms, using extraction methods such as membrane-based solid phase extraction and magnetic bead-based extraction. Among these methods, magnetic bead-based solid phase extraction is widely used for integrated nucleic acid-based molecular diagnostic platforms due to its speed, simplicity, and seamless integration with microfluidic technology. However, most extraction approaches depend on traditional PCR technology or single isothermal amplification assays, increasing the cost of instruments for precise temperature control or potentially causing false-positive signals due to non-specific amplification.

Recently, CRISPR technology has emerged as a powerful tool for nucleic acid-based molecular detection due to its simplicity, robustness, and high specificity. In particular, researchers have developed several highly sensitive and specific CRISPR-based molecular diagnostic platforms, such as specific high-sensitivity enzymatic reporter unlocking (SHERLOCK) and DNA endonuclease-targeted CRISPR trans reporter (DETECTR), by combining CRISPR detection with isothermal amplification technologies (e.g., recombinase polymerase amplification [RPA]). However, most of these CRISPR assays are limited to detecting purified nucleic acid samples and lack a “sample-to-result” detection capacity. Typically, nucleic acid extraction and purification methods require bulky instruments (e.g., centrifuge machines) and multiple manual operations, which is not ideal for point-of-care diagnostic applications.

The present disclosure addresses this need by providing a simple, affordable, portable, and sensitive LIAMT platform for the “sample-to-result” nucleic acid detection at the point-of-care. Certain aspects of the compositions and methods of the disclosure are described in more detail below.

The following terms are used to describe the invention of the present disclosure. In instances where a term is not specifically defined herein, that term is given an art-recognized meaning by those of ordinary skill applying that term in context to its use in describing the present disclosure.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

The term “sample” is used herein in the broadest sense and refers to any suitable sample, including liquids, solids, and gases. In some embodiments, the sample is a biological sample (e.g., a sample obtained from a subject). The biological sample may comprise a fluid sample or a tissue sample. In some embodiments, the biological sample is a blood sample or a blood product such as serum or plasma. In some embodiments, the sample comprises urine. In embodiments, the sample is a respiratory specimen, including a nasal sample (e.g., a nasal swab), a nasopharyngeal sample (e.g., a nasopharyngeal swab), an oropharyngeal sample (e.g., an oropharyngeal swab), a mid-turbinate sample (e.g., a midturbinate swab), sputum, endotracheal aspirate or bronchoalveolar lavage. In some embodiments, the sample is a cerebrospinal fluid sample. In some embodiments, the sample is a saliva sample. In some embodiments, the sample is a tissue sample. In some embodiments, the sample is obtained from a subject suspected of having a viral infection (e.g., HIV). In some embodiments, the sample is obtained from a subject suspected of having an upper respiratory infection. In some embodiments, the sample is obtained from a subject suspected of having a SARS-CoV-2 infection. In some embodiments, the subject is a human. The sample can be used directly as obtained from a patient or can be pre-treated, such as by heating, filtration, distillation, extraction, concentration, centrifugation, inactivation of interfering components, addition of reagents, and the like.

The terms “target sequence,” “target nucleic acid,” and “target site” are used interchangeably herein to refer to a polynucleotide (nucleic acid, gene, chromosome, genome, etc.) to which a guide sequence (e.g., a guide RNA) is designed to have complementarity, wherein hybridization between the target sequence and a guide sequence promotes the formation of a Cas/CRISPR complex, provided sufficient conditions for binding exist. In some embodiments, the target sequence is a viral nucleic acid sequence. In some embodiments, the target sequence is a SARS-CoV-2 sequence. In some embodiments, the target sequence is a HIV sequence. In an embodiment, the target sequence includes several types of nucleic acids such as miRNA, mRNA, circulating cell-free DNA (cfDNA), RNA (cfRNA), and/or a combination thereof.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The terms “crRNA” or “CRISPR RNA” are used interchangeably herein. The term crRNA is used in the broadest sense to cover any RNA involved in CRISPR methods, including pre-crRNA, tracrRNA, and guide RNA.

The “guide RNA,” “single guide RNA,” and “synthetic guide RNA,” are used interchangeably herein and refer to a nucleic acid comprising a crRNA containing a guide sequence. The terms “guide sequence,” “guide,” and “spacer,” are used interchangeably herein and refer to the about 20 nucleotide sequence within a guide RNA that specifies the target site. In CRISPR/Cas systems, the guide RNA contains an approximate 20-nucleotide guide sequence followed by a protospacer adjacent motif (PAM) that directs the endonuclease via Watson-Crick base pairing to a target sequence.

As used herein, a “nucleic acid” or a “nucleic acid sequence” refers to a polymer or oligomer of pyrimidine and/or purine bases, preferably cytosine, thymine, and uracil, and adenine and guanine, respectively. The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid, locked nucleic acid (LNA), cyclohexenyl nucleic acids, and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein,” are used interchangeably herein. [0048] As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

As used herein, the term “substantially” means to a great or significant extent, but not completely.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. All ranges disclosed herein include both end points as discrete values as well as all integers and fractions specified within the range. For example, a range of 0.1-2.0 includes 0.1, 0.2, 0.3, 0.4 . . . 2.0. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” means the actual physical introduction of a composition into or onto (as appropriate) a subject, a host, or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein. “Providing” means giving, administering, selling, distributing, transferring (for profit or not), manufacturing, compounding, or dispensing.

As used herein, “optional” or “optionally” means that the subsequently described event or circumstance may or may not occur, and that the description includes instances where said event or circumstance occurs and instances where it does not.

The term “subject” or “patient” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject or patient is a human subject or a human patient, such as a human being treated or assessed for a disease, disorder or condition, a human at risk for a disease, disorder or condition, a human having a disease, disorder or condition, and/or human being treated for a disease, disorder or condition as described herein. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included. As used herein, a subject is “in need of treatment” if such subject would benefit biologically, medically, or in quality of life from such treatment. A subject in need of treatment does not necessarily present symptoms, particular in the case of preventative or prophylaxis treatments.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

A significant change is any detectable change that is statistically significant in a standard parametric test of statistical significance such as Student's t-test, where p<0.05.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

In an aspect, disclosed is a system including lab-in-a-magnetofluidic tube (LIAMT) platform for molecular detection of virus in a sample. The LIAMT platform includes a LIAMT device (e.g., disposable device) and a sample processor/detector (e.g., reusable processor/detector). In an embodiment, the reusable processor/detector is a palm-sized processor/detector. In an embodiment, the system is a self-contained, all-in-one, microfluidic device that leverages the magnetofluidic transport of micro/nano magnetic beads, enabling “sample-to-result” nucleic acid testing. In one exemplary embodiment, the system can perform viral lysis, nucleic acid extraction, amplification (e.g., reverse transcription RPA (RT-RPA)) and detection (e.g., CRISPR-based molecular detection). To enable simple point-of-care molecular diagnostics, the processor such as a palm-sized processor was developed for magnetofluidic operation, isothermal amplification, and visual fluorescence detection. In an embodiment, the detection signals can be visually observed by the naked eye or recorded by a smartphone, eliminating the need for an expensive optical instrument. In an embodiment, the system is applied to detect pathogen nucleic acids, such as viral nucleic acids, for example SARS-CoV-2 and HIV. To demonstrate its clinical utility, the system was utilized to detect these viruses in clinical samples, thus demonstrating great promise for simple, sensitive, and affordable point-of-care diagnostics of infectious pathogens in resource-limited settings or even at home.

Exemplary devices, systems, kits, assays, reagents, and methods are described herein.

### Devices

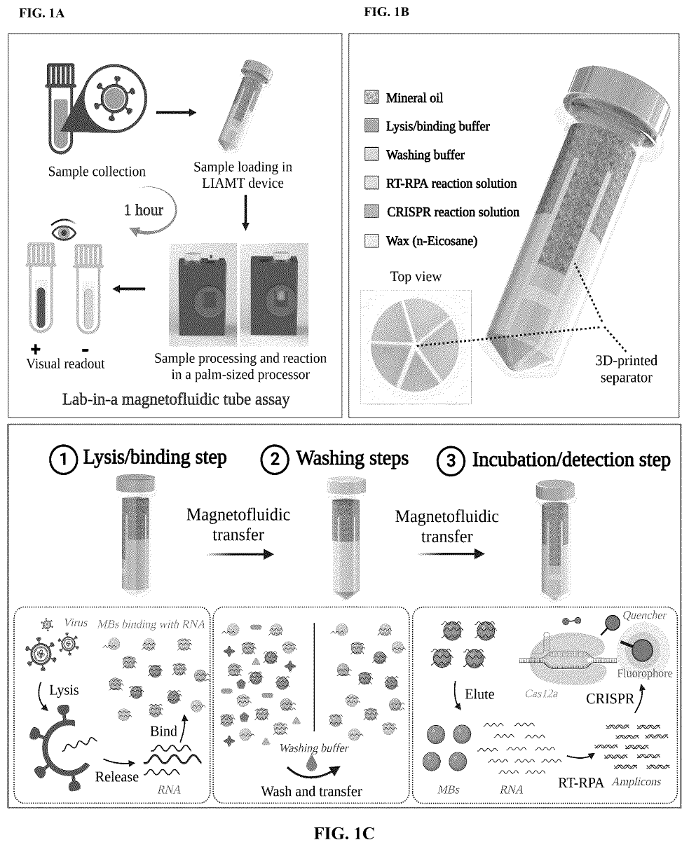

In an aspect, disclosed is a LIAMT device. Exemplary devices are shown in FIGS. 1A and B. For example, in some embodiments, the device is a reaction vessel (e.g., reaction tube) comprising a plurality of distinct assay zones or chambers comprising one or more of a) a lysis zone comprising a lysis/binding buffer and magnetic beads; b) one or more (e.g., 1, 2, 3, or more) washing zones comprising a washing buffer; c) an amplification zone comprising amplification reagents; and d) a detection zone comprising nucleic acid detection reagents.

The present disclosure is not limited to particular configurations of the reaction vessel. In some embodiment, the assay zones are vertically oriented columns in the reaction vessel. In some embodiments, the top of the reaction vessel is covered with mineral oil in fluid contact with the top surface of each of the assay zones.

In certain aspects, the amplification zone and the detection zone are in the same assay zone and are separated by temperature-sensitive wax (e.g., n-Eicosane wax).

In some embodiments, the device further comprises a blood sample zone comprising a plasma separation membrane for separation of plasma from blood (e.g., for detection of target nucleic acids directly from blood). The present disclosure is not limited to particular plasma separation membranes. In some embodiments, commercially available membranes are utilized (e.g., from Cytiva, Marlborough MA or Thermo-Fisher, Waltham, MA).

In an embodiment, the reaction vessel includes a lysis zone with about 60-120-degree angle, for example, in some embodiments, the lysis zone comprises about 65°, 70°, 75°, 80°, 85°, 90°, 95°, 100°, 105°, 110°, or about 115° angle.

In an embodiment, the sample tube includes 1, 2, or 3 three wash zones with about 60-90-degree angle each, for example, three wash zones with about 65°, 70°, 75°, 80°, 85°, or about 90° angle. In an embodiment, the sample tube includes an amplification/wax/detection chamber with about 30-90-degree angle, for example, the amplification/wax/detection chamber with about 35°, 40°, 45°, 50°, 55°, 60°, 65°, 70°, 75°, 80°, or about 85° angle. The chamber angle decides the volumes of each chamber, the bigger the chamber angle, the larger its volume. It is possible to get the same results with different angles by modifying the components of lysis/binding buffer and wash buffer.

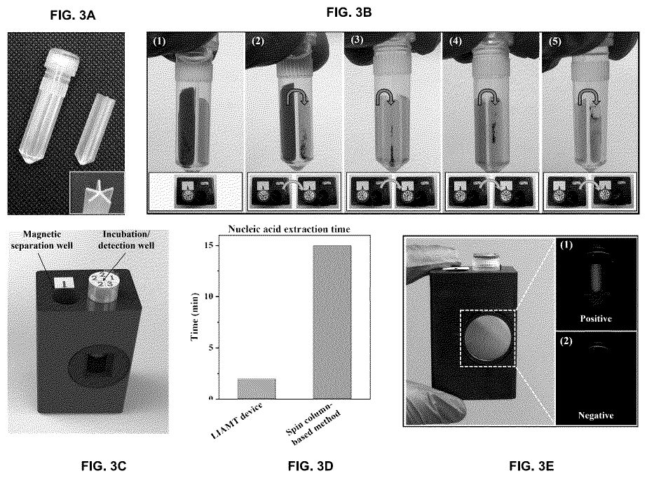

In an embodiment, the chambers or zones are separated by a separator. An example of a separator is shown in FIG. 3A. As shown in FIG. 3A, in some embodiments, the separator comprises vertically oriented thin sheets joined at the center, although other configurations of separators are specifically contemplated. In an embodiment, the separator is a 3D-printed separator.

The separator can be fabricated from or comprise any suitable material that is resistant to buffers and reagents used herein. In an embodiment, the 3D-printed separator is washed with a solvent (e.g., isopropyl alcohol) for at least 30 min and cured under ultraviolet (UV) light for at least 3 hours. In an embodiment, after UV curing, the edges of the separator were coated with polydimethylsiloxane (PDMS) solution. In an embodiment, the assembled LIAMT devices were placed at room temperature for more than 12 hours, allowing the PDMS to completely solidify.

As described above, the lysis zone comprises a plurality of magnetic beads or particles. In an embodiment, the surface of the magnetic beads is coated with appropriate functional groups, which can bind or adsorb nucleic acids. In an embodiment, the functional groups of magnetic beads for nucleic acids binding are carboxyl groups, hydroxyl groups, silicon groups, or a combination thereof.

Any number of suitable buffers and assay reagents may be utilized in the devices described herein. For example, in some embodiments, the wash buffer is MgOAc (e.g., 50 mM MgOAc) with 1% Triton-X-100. In some embodiments, the amplification reagents are reagents for isothermal amplification (e.g., recombinase polymerase amplification (RPA) or reverse transcriptase RPA (RT-RPA)) of a target nucleic acid (e.g., nucleic acid primers, nucleotides, buffer, and/or polymerases). Additional assays and reaction mixtures for use in the devices are described below.

### Systems

The present disclosure also provides systems comprising a device as described herein and a sample processor. In some embodiments, the sample processor comprises a magnetofluidic separation well for magnetofluidic transferring and operation, and an incubation/detection well.

In an embodiment, the sample processor includes two wells: i) a magnetofluidic separation well for magnetofluidic transferring and operation, and ii) an incubation/detection well for amplification and detection (e.g., fluorescence detection).

In additional embodiments, the sample processor includes a single well configured for performing both i) magnetofluidic separation, and ii) incubation/detection (e.g., fluorescence detection).

The magnetofluidic separation well utilizes a magnet to transfer magnetic beads or particles bound to target nucleic acids between the lysis zone, the washing zone(s), and the amplification and detection zone. The present disclosure is not limited to particular magnets. In some embodiments, the magnet is a cylindrical neodymium magnet (about 6.33 mm diameter×about 6.33 mm height) that is embedded to separate the magnetic beads and transfer them into different functional zones of the device through magnetic force. Any suitable magnet can be used to separate the magnetic beads and transfer them into different functional zones of the device through magnetic force.

In an embodiment, the LIAMT device moves substantially only magnetic particles including bound biomolecules among the chambers or wells. In an embodiment, the magnetic particles are transferred from one chamber to another chamber by neodymium magnet outside the LIAMT device. For example, in some embodiments, a user lifts the device and rotates the device, allowing the magnet in the magnetofluidic separation well to transfer magnetic beads between zones. In other embodiments, a stepper motor is used to lift and rotate the device in order to move magnetic beads bound with target nucleic acids between assay zones.

In an embodiment, the incubation/detection well includes a flexible thin-film heater and a thermocouple to heat the device and to monitor the temperature. In an embodiment, a Cy5 fluorescence filter set and LED lights are used to build an optical detection path to visualize the fluorescence signal of the LIAMT device. In an embodiment, an LED light is placed at the bottom of the incubation/detection well and to serve as an excitation light source. In an embodiment, an excitation filter is used to cover the LED to block light with other wavelengths. In an embodiment, an emission filter is placed in the observation window of the processor to obtain clear fluorescence signal from the LIAMT device. In an embodiment, the sample processor is a palm-sized processor.

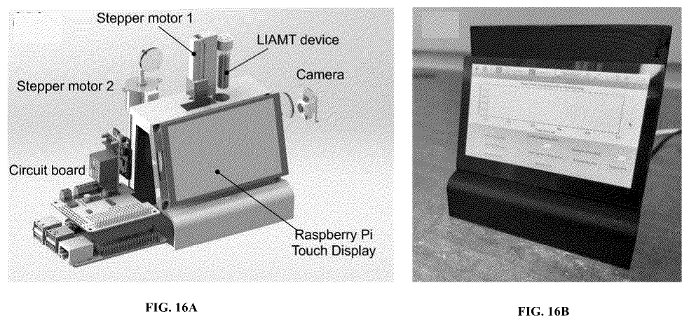

As described in FIGS. 16A-B, in some aspects, a fully automated device comprising a microprocessor (e.g., Raspberry Pi microprocessor) is utilized. The device comprises, in some aspects, a circuit board, microprocessor, one or more (e.g., two) stepper motors with motor drivers, camera, and fluorescence filter. In some embodiments, the stepper motors comprise plurality of first stepper motors configured to transfer magnetic beads within assay zones of the device and a plurality of second stepper motors configured to move the device (e.g., between the magnetofluidic separation well and the incubation/detection well or into and out of a single magnetofluidic separation and incubation/detection well).

In some embodiments, the device automates sample processing, detection, and display of results. In some embodiments, the microprocessor is configured to real-time monitor signal (e.g., fluorescence signals), analyze the fluorescence images and quantify the nucleic acid target.

### Reagents and Assays

The present disclosure is not limited to particular amplification and detection assays. In the below discussion, the disclosure is exemplified with RT-RPA amplification and CRISPR detection. However, any number of suitable amplification and detection methodologies can be employed with the devices, systems, and methods described herein.

In an embodiment, the target is amplified by isothermal amplification. In an embodiment, the assays described herein take advantage of isothermal amplification techniques to amplify the target, if present in the sample, and CRISPR/Cas technology to detect the target, if present. Accordingly, in an embodiment, the CRISPR reaction mixture comprises a Cas endonuclease and a target-specific crRNA. In an embodiment, the Cas endonuclease may be Cas9, Cas 12a (Cpf1), or Cas13. In an embodiment, the endonuclease is Cas12a. The target-specific crRNA depends on the intended target to be detected in the sample. Without wishing to be bound by this theory, it is believed that the Cas endonuclease (e.g., Cas 12a) and the target-specific crRNA form a complex. For example, the Cas12a and the target specific crRNA forms a complex, referred to as a Cas12a-crRNA complex. The complex binds to a target sequence on a nucleic acid, and induces selective cleavage, which generates a fluorescent signal that can be detected and/or measured to determine the presence of the target in the sample. In an embodiment, the crRNA comprises a guide RNA sequence (gRNA) that directs the endonuclease-crRNA complex (e.g., the Cas12a-crRNA complex) to bind to the desired sequence.

In an embodiment, the target is a nucleic acid. In an embodiment, the target is a nucleic acid from a pathogen. In an embodiment, the target is a viral nucleic acid. In an embodiment, the target may be a viral RNA. In an embodiment, the target includes a viral nucleic acid from a virus causing upper respiratory infection. In an embodiment, the target includes a viral nucleic acid from an upper respiratory pathogen selected from SARS-CoV2, coronavirus, rhinovirus, influenza, respiratory syncytial virus, adenovirus, parainfluenza, human immunodeficiency virus, human papillomavirus, rotavirus, hepatitis C virus, zika virus, Ebola virus, tuberculosis, Borrelia burgdorferi, Staphylococcus, aspergillus, Streptococcus, pyogenes, or a combination thereof. In some embodiments, the target is SARS-CoV-2 nucleic acid. In an embodiment, the target sequence includes SARS-CoV-2, influenza virus, drug-resistant influenza viruses, human immunodeficiency virus (HIV), and/or a combination thereof. In an embodiment, the target sequence includes several types of biomarkers such as miRNA, mRNA, circulating cell-free DNA (cfDNA), RNA (cfRNA), and/or a combination thereof. In an embodiment, the target sequence is a SARS-CoV-2 sequence. In an embodiment, the target sequence is a human immunodeficiency virus (HIV).

In an embodiment, the CRISPR reaction mixture further includes primers for amplification of the target. In an embodiment, the CRISPR reaction mixture includes primers for isothermal amplification of the target. In an embodiment, the CRISPR reaction mixture includes primers for dual-priming isothermal amplification of the target (DAMP). In an embodiment, the CRISPR reaction mixture includes outer primers and inner primers. In an embodiment, the CRISPR reaction mixture includes a forward outer primer and a reverse outer primer. In an embodiment, the outer primers are 10-40 contiguous nucleotides in length. For example, the forward outer primer and/or the reverse outer primer may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous nucleotides in length. In an embodiment, the CRISPR reaction mixture further includes a forward inner primer and reverser inner primer. In an embodiment, the inner primers are 20-60 contiguous nucleotides in length. For example, in an embodiment, the forward inner primer and/or the reverse inner primer are 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 contiguous nucleotides in length.

In an embodiment, the primers are designed to amplify the target (e.g., the target nucleic acid sequence). Accordingly, appropriate selection of primers depends on the target itself. In an embodiment, the target is SARS-CoV-2. In an embodiment, the target is or is present within the nucleoprotein (N) gene of SARS-CoV-2. Accordingly, the primers may be designed to amplify the desired portion of the N gene of SARS-CoV-2. Other suitable primers may be designed for amplification of other desired targets. For example, the target may be viral nucleic acid, and the primers are designed for amplification of said viral nucleic acid. For example, primers may be designed and used for amplification of viral nucleic acid selected from SARS-CoV-2, coronavirus, rhinovirus, influenza, respiratory syncytial virus, adenovirus, parainfluenza, human immunodeficiency virus (HIV), human papillomavirus, rotavirus, hepatitis C virus, zika virus, Ebola virus, tuberculosis, Borrelia burgdorferi, Staphylococcus, Aspergillus, Streptococcus Pyogenes nucleic acid, or a combination thereof.

In an embodiment, the CRISPR reaction mixture includes a forward outer primer and a reverse outer primer. In an embodiment, the CRISPR reaction mixture includes inner primers such as a forward inner primer and/or a reverse inner primer. In an embodiment, the forward inner primer and/or the reverse inner primer is phosphorothioated. In an embodiment, the forward inner primer and the reverse inner primer is phosphorothioated. A “phosphorothioated primer” or a primer that “is phosphorothioated” refers to a primer in which at least one nucleotide comprises a phosphorothioate modification. For example, a phosphorothioated primer may comprise at least one, at least two, at least three, at least four, at least five, at least six, at least seven, at least eight, at least nine, or at least 10 nucleotides that are phosphorothioated. In an embodiment, the nucleotides that are phosphorothioated are contiguous. For example, the primer may contain at least two, at least three, at least four, at least five, at least six, at least seven, at least eight, at least nine, or at least 10 contiguous nucleotides that are phosphorothioated. In an embodiment, a phosphorothioated primer includes 11, 12, 13, 14, 15, 16, 17, 18, 19, 20, 21, 22, 23, 24, 25, 26, 27, 27, 29, or 30 nucleotides that are phosphorothioated.

In an embodiment, the CRISPR reaction mixture further includes a forward competition primer and/or a reverse competition primer. The competition primers may be added to mediate pair-priming strand extension. In an embodiment, the competition primers overlap with the inner primers, but are shorter than the inner primers. For example, in an embodiment, the forward competition primer includes at least 10 contiguous nucleotides also present within the sequence of the forward inner primer, wherein the forward competition primer is shorter than the forward inner primer. For example, in an embodiment, the forward competition primer includes 10, 11, 12, 13, 14, 15, 16, 17, 18, 19, 20, 21, 22, 23, 24, 25, 26, 27, 28, 29, or 30 contiguous nucleotides also present within the sequence of the forward inner primer. In an embodiment, the forward competition primer comprises 15-25 contiguous nucleotides present within the nucleotide sequence of the forward inner primer.

In an embodiment, the reverse competition primer comprises at least 10 contiguous nucleotides also present within the sequence of the reverse inner primer, wherein the reverse competition primer is shorter than the reverse inner primer. For example, in an embodiment, the reverse competition primer includes 10, 11, 12, 13, 14, 15, 16, 17, 18, 19, 20, 21, 22, 23, 24, 25, 26, 27, 28, 29, or 30 contiguous nucleotides also present within the sequence of the reverse inner primer. In an embodiment, the reverse competition primer includes 15-25 contiguous nucleotides present within the nucleotide sequence of the reverse inner primer.

In an embodiment, the CRISPR reaction mixture further includes pyrophosphatase. In an embodiment, the CRISPR reaction mixture further includes a DNA polymerase. In an embodiment, the DNA polymerase comprises Bst DNA polymerase. In an embodiment, the CRISPR reaction mixture further comprises a reverse transcriptase.

In an embodiment, the CRISPR-reaction mixture additionally comprises a reporter molecule. In an embodiment, the reporter molecule generates a signal, wherein the signal is detected to determine the presence and/or amount of target within the sample. For example, in an embodiment, the reporter molecule includes a fluorescent moiety conjugated to a quencher. In an intact (e.g., uncleaved) state, the fluorescence from the fluorescent moiety is quenched, such as by resonance energy transfer. However, after cleavage of the reporter molecule, the fluorescent moiety is released from the quencher and the fluorescent signal becomes detectable. In an embodiment, the reporter molecule includes a single stranded DNA reporter. In an embodiment, the reporter molecule includes a single-stranded, non-target DNA sequence labeled with a fluorescent label. For example, the reporter molecule may include a single-stranded, non-target DNA sequence labeled with fluorescein or derivatives thereof (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like). The label may be present at the 5′ end or the 3′ end of the non-target DNA sequence.

In an embodiment, the endonuclease indiscriminately cleaves reporter molecules during cleavage of the amplicon generated as a result of isothermal amplification. Accordingly, binding of the Cas-crRNA complex (e.g., Cas12a-crRNA complex) to the target amplicon induces cleavage of the reporter molecule. In contrast, in the absence of the target amplicon the endonuclease (e.g., Cas12a) does not cause substantial (e.g., detectable) cleavage of the reporter molecule.

The CRISPR reaction mixture may further include additional reagents. For example, the CRISPR reaction mixture may include additional reagents to stabilize the sample (e.g., preservatives, inhibitors, etc.), along with suitable buffers, salts, dNTPs, and the like required for isothermal amplification and detection of the target, if present in the sample.

In an aspect, provided herein is an assay for detection of a nucleic acid (e.g., DNA or RNA) target such as virus in a sample. In an embodiment, the assay includes virus lysis, nucleic acid extraction, and CRISPR-powered molecular detection for detecting a target in the sample. In an embodiment, the assay includes a fully integrated lab-in-a magnetofluidic tube (LIAMT) platform. In an embodiment, the assay for detecting a target in a sample includes contacting a sample with a CRISPR reaction mixture, partitioning CRISPR reaction mixture into a plurality of zones or chambers such as microwells, amplifying the target, if present in the sample, and detecting a signal in each of the plurality of chambers. In an embodiment, detection of the signal in a given chamber indicates the presence of the target in the chamber. In an embodiment, the sample is from a subject. In an embodiment, the subject is a mammal such as human. In some embodiments, the subject is a mammal such as, for example, a dog, cat, cattle, or pig.

### Methods

Embodiments of the disclosure provide method detecting a target such as an nucleic acid sequence in a virus in a sample with a LIAMT device, the method including: a) contacting a sample with a CRISPR reaction mixture including a lysis/binding buffer zone for viral lysis and nucleic acid binding on the magnetic beads; b) washing buffer zones for nucleic acid purification, and a CRISPR reaction zone; c) partitioning the CRISPR reaction mixture into a plurality of zones or chambers (such as compartments, wells, or microwells); d) amplifying the target, if present in the sample, by isothermal amplification; and e) detecting a signal in the plurality of zones (such as compartments, wells, or microwells), wherein detection of the signal in a given zone indicates the presence of the target in the sample. In an embodiment, the sample is from a subject. In an embodiment, the subject is a mammal such as human. In some embodiments, the subject is a mammal such as, for example, a dog, cat, cattle, or pig.

In an embodiment, the CRISPR reaction mixture has been contacted with the sample prior to partitioning. In an embodiment, the sample is present within the chambers. The method further includes amplifying the target, if present in the sample, by isothermal amplification. In an embodiment, isothermal amplification comprises heating (or incubating) the CRISPR reaction mixture at a temperature of about 30° C.-60° C. for at least about 10 minutes. For example, isothermal amplification may include heating at a temperature of about 30° C.-50° C. for at least about 30 minutes, at least about 40 minutes, at least about 50 minutes, at least about 60 minutes, at least about 70 minutes, at least about 80 minutes, or at least about 90 minutes. In an embodiment, isothermal amplification includes heating/incubating at a temperature of about 35° C.-50° C. for about 60 minutes. In an embodiment, the temperature is about 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C. In an embodiment, the temperature is about 37° C. In an embodiment, the temperature is about 45° C. In an embodiment, isothermal amplification includes heating/incubating at about 37° C. for about 30 minutes followed by heating/incubating at about 45° C. for about 30 minutes.

The sample may be any suitable sample. In an embodiment, the sample is a biological sample (e.g., a sample obtained from a subject). The biological sample may include a fluid sample or a tissue sample. In an embodiment, the biological sample is a blood sample or a blood product such as serum or plasma. In an embodiment, the sample includes urine. In an embodiment, the sample is a respiratory specimen, including a nasal sample, an oropharyngeal sample, a mid-turbinate sample, sputum, endotracheal aspirate or bronchoalveolar lavage. In an embodiment, the sample is a cerebrospinal fluid sample. In an embodiment, the sample is a saliva sample. In an embodiment, the sample is a tissue sample. In an embodiment, the sample is obtained from a subject suspected of having a viral infection. In an embodiment, the sample is obtained from a subject suspected of having a SARS-CoV-2 infection. In an embodiment, the sample is obtained from a subject suspected of having a HIV infection. In an embodiment, the subject is a human. The sample can be used directly as obtained from a subject/patient or can be pre-treated, such as by heating, filtration, distillation, extraction, concentration, centrifugation, inactivation of interfering components, addition of reagents, and the like.

In some embodiments, the sample is a research or screening sample not derived from a patient or subject. For example, in some embodiments, the sample is a sample with a known amount of nucleic acid. Such samples can be used in research or screening settings, or as standards or calibrators for detection assays.

In certain aspects, assays are performed in a clinical laboratory, at a point of care (e.g., medical clinic or hospital) or at home (e.g., by a subject or patient).

### Kits

In an aspect, disclosed are kits. In an embodiment, provided herein is a kit including the system as shown and described herein including one or more primers, compositions, or CRISPR reaction mixtures described herein. In an embodiment, the kit includes the lab-in-a magnetofluidic tube (LIAMT) platform/device as shown and described herein, including a processor. In an embodiment, the kit further includes reagents for CRISPR-based detection of the isothermally amplified nucleic acid. For example, the kit may further include a reporter molecule. In an embodiment, the reporter molecule includes a single-stranded, non-target DNA sequence labeled with a fluorescent label. For example, the reporter molecule may include a single-stranded, non-target DNA sequence labeled with fluorescein or derivatives thereof (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like). The label may be present at the 5′ end or the 3′ end of the non-target DNA sequence.

The kit may be used for methods of detecting a target in a sample. In an embodiment, the target is nucleic acid (e.g., RNA or DNA). In an embodiment, the target is viral nucleic acid. For example, the target may be nucleic acid from an upper respiratory pathogen. For example, the kit may be used in methods of detecting SARS-CoV-2 nucleic acid in a sample. The kit may include additional components, including tubes (e.g., sample collection tubes, storage tubes, reaction tubes), buffers, stabilizers, salts, controls, calibrators, and the like. The kit may additionally include instructions for use. Instructions included in kits can be affixed to packaging material or can be included as a package insert. While the instructions are typically written or printed materials, they are not limited to such. Any suitable medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure is illustrated and further described in more detail with reference to the following non-limiting examples. Section headings as used in this section and the entire disclosure herein are merely for organizational purposes and are not intended to be limiting.

### Example 1

### Overview of LIAMT Platform

FIG. 1A summarizes the clinical assay workflow of the LIAMT platform. The LIAMT platform includes a disposable, all-in-one LIAMT device and a palm-sized processor (FIG. 1A). The LIAMT device can directly accept raw clinical samples (e.g., swab, plasma) and perform “sample-to-result” molecular diagnostics within ˜1 hour. For clinical testing, the collected sample is first added to the LIAMT device. Then, the device is inserted into the magnetic separation well of the palm-sized processor for magnetofluidic-based nucleic acid purification. After nucleic acid purification, the device is transferred into the incubation/detection well of the processor for isothermal amplification and CRISPR detection. Last, the endpoint fluorescence signal of the device can be visually read by the naked eye or recorded by a smartphone, eliminating the need for time-consuming nucleic acid sample preparation and expensive instrumentation (FIG. 1A).

FIG. 1B shows a schematic illustration of the fully integrated and self-contained LIAMT device, which is capable of performing nucleic acid sample preparation, isothermal amplification, CRISPR cleavage reaction, and fluorescence detection in a single engineered tube. The disposable LIAMT device is composed of a three-dimensional (3D)-printed separator and a commercially available 1.5-mL microcentrifuge tube. The separator isolates the microcentrifuge tube into five independent zones: i) one lysis/binding buffer zone for viral lysis and nucleic acid binding on the magnetic beads, ii) three washing buffer zones for nucleic acid purification, and iii) one RT-RPA/wax (n-Eicosane)/CRISPR reaction zone (inset of FIG. 1B). All buffer solutions and reagents are pre-stored in the LIAMT device and covered by mineral oil. As shown in FIG. 1C, the virus is first lysed to release nucleic acids when the clinical sample is mixed with the pre-stored lysis/binding buffer in the lysis/binding buffer zone. The released nucleic acid can bind the surface of the magnetic beads in the buffer solution due to electrostatic interactions. Then, the magnetic beads bound with nucleic acids are transferred to the washing buffer zones to remove potential inhibitors by the magnetofluidic separation approach. Next, the magnetic beads carrying nucleic acids are transferred to the RT-RPA/wax/CRISPR reaction zone for isothermal amplification and CRISPR-based fluorescence detection. Thus, the LIAMT platform provides a simple, rapid, and affordable “sample-to-result” solution to detect virus in clinical samples at the point of care.

### Optimization of Magnetofluidic-Based Nucleic Acid Sample Preparation.

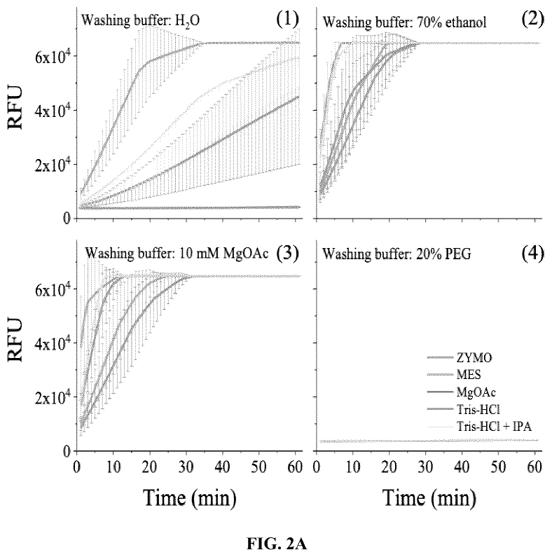

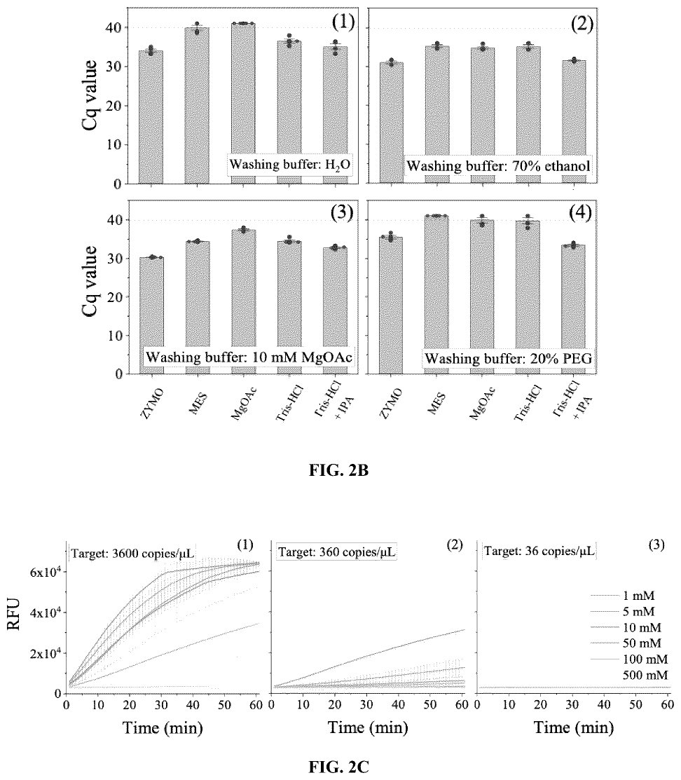

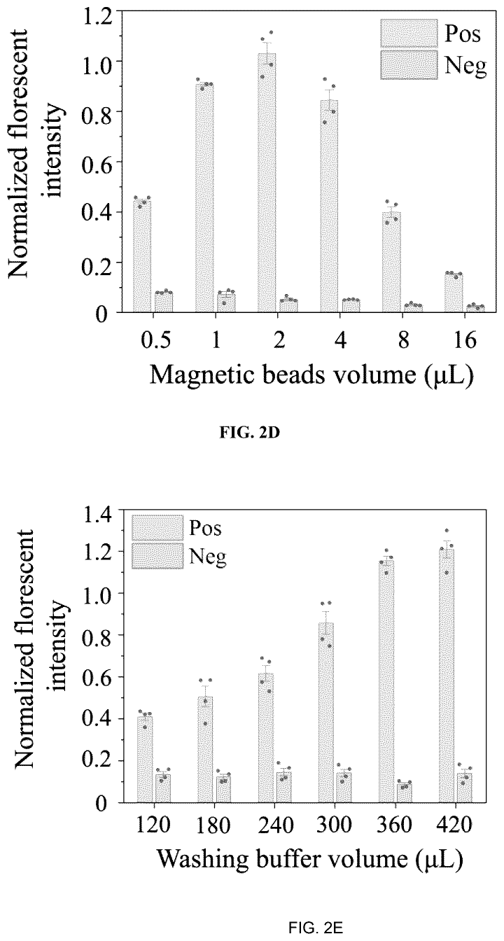

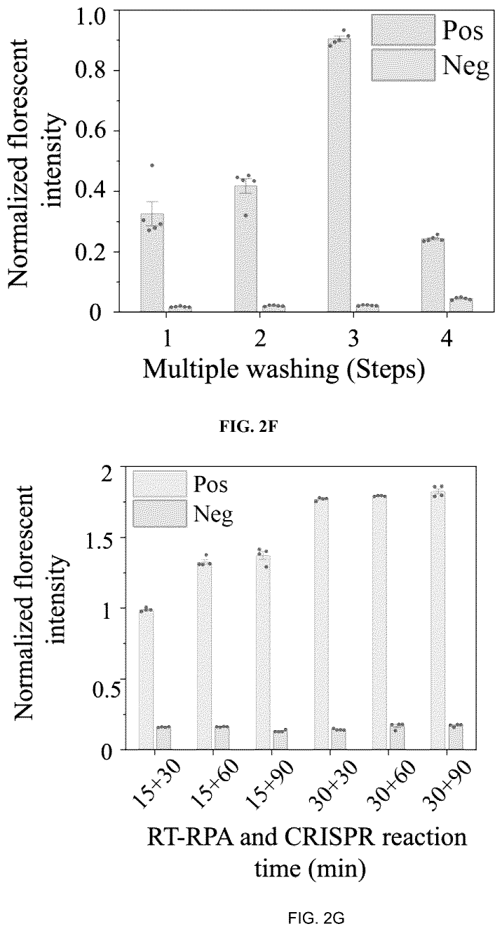

To prepare high-quality nucleic acid samples, different lysis/binding buffer and washing buffer solutions for the magnetofluidic-based nucleic acid extraction were compared and optimized. First, five different lysis/binding buffer solutions were compared, including: i) ZYMO lysis/binding buffer (ZYMO Research), ii) MES buffer, iii) MgOAc buffer, iv) Tris-HCl buffer, and v) Tris-HCl+isopropanol buffer. In addition, four different washing buffer solutions were evaluated, including: i) deionized water, ii) 70% ethanol, iii) MgOAc with 1% Triton X-100, and iv) 20% polyethylene glycol (PEG) solution. As shown in FIG. 2A, the combination of the ZYMO lysis/binding buffer and MgOAc washing buffer with 1% Triton X-100 showed the best performance in the RT-RPA/CRISPR assay. To further validate its versatility for other nucleic acid amplification testing, extracted nucleic acids were tested by using an RT-PCR assay and similar results as the RT-RPA/CRISPR assay were obtained (FIG. 2B). Next, the effect of the MgOAc concentration in the washing buffer on the nucleic acid extraction and downstream amplification detection was evaluated by comparing and testing six different concentrations (1, 5, 10, 50, 100, and 500 mM) of MgOAc buffer with 1% Triton X-100 by various SARS-CoV-2 concentration samples in the LIAMT device. To monitor the fluorescence signal of the CRISPR reaction in real time, the RPA amplicons of each tested sample from the device were removed and added to the CRISPR reaction tubes for real-time fluorescence monitoring. As shown in FIG. 2C, the washing buffer of 50 mM MgOAc consistently showed the highest fluorescence signal for SARS-CoV-2 detection. Thus, the optimal lysis/binding buffer and washing buffer for the LIAMT device was determined to be ZYMO lysis/binding buffer and 50 mM MgOAc washing buffer with 1% Triton X-100, respectively.

Unlike conventional magnetic bead-based nucleic acid extraction methods (12-14), the LIAMT assay retains the magnetic beads in the RT-RPA reaction solution, eliminating the need for an additional elution step. Thus, the usage amount of the magnetic beads can affect the downstream nucleic acid detection. To this end, a series of volumes of magnetic beads ranging from 0.5 to 16 μL was tested in the LIAMT device. The optimal volume of the magnetic beads was determined to be 2 μL, as it led to the strongest fluorescence signals (FIG. 2D). As the usage of the magnetic beads increased, there was a significant reduction in the fluorescence signals of the device. This result could be attributed to two major factors: i) the increased amount of magnetic beads may introduce more potential inhibitors into the downstream RT-RPA reaction solution, and ii) an excessive amount of magnetic beads can potentially interfere with the RT-RPA reaction and reduce its amplification efficiency. In addition, the effect of the washing buffer volume and washing times on the LIAMT assay was evaluated. Various volumes of the washing buffer ranging from 120 to 420 μL were tested. Increasing the volume of the washing buffer resulted in a higher fluorescence detection signal (FIG. 2E). Considering the limited volume capacity of the LIAMT device, 420 μL of washing buffer was used to wash the magnetic beads and purify the nucleic acids. To optimize the washing efficiency, the number of washing steps was investigated by evenly dividing 420 μL of washing buffer into 1, 2, 3, and 4 equal parts. As the number of washing times was increased, an increased fluorescence signal (FIG. 2F) was observed. However, when more than three washing steps were used, the detection signals dramatically reduced, which may be attributed to the increased loss of magnetic beads during the magnetofluidic transferring process. Therefore, 2 L of magnetic beads and 420 μL of washing buffer with three washing steps was selected for subsequent experiments.

Lastly, the effect of the incubation time of the RT-RPA reaction and CRISPR reaction for nucleic acid amplification and detection was tested. To maximum the efficiency of the RT-RPA reaction, the RT-RPA preamplification independently occurs in the RT-RPA/wax/CRISPR zone of the device. This separation occurs because its reaction mixture is initially isolated by the wax from the CRISPR reaction solution. Thus, reaction times were tested and assayed. As shown in FIG. 2G, a 30-min RT-RPA preamplification reaction and 30-min CRISPR reaction generated the strongest fluorescence signal for detection. Increasing the CRISPR reaction time did not significantly improve the fluorescence signal. Thus, a 30-min RT-RPA preamplification reaction and 30-min CRISPR reaction was chosen for use in the LIAMT device.

### Design and Operation of LIAMT Device.

After optimizing the experimental conditions of the magnetofluidic-based nucleic acid detection, a disposable, all-in-one, LIAMT device for “sample-to-result” detection of virus was designed, fabricated, and tested. 3D-printing technology was used to fabricate the separator (FIG. 3A) because of its low cost and ease of use. For clinical testing, raw clinical samples were directed added into the lysis/binding buffer zone to lyse the virus and release nucleic acids (FIG. 3B). Thus, the released nucleic acid binds the surface of the silica-coated magnetic beads due to electrostatic interactions. After inserting the device into the magnetic separation well of the processor (FIG. 3C), the magnetic beads are transferred to the washing buffer zones for nucleic acid purification by manually rotating and lifting the device. To minimize the potential inhibitor's interference and obtain high-quality nucleic acids, the separator was designed with three buffer zones to wash the magnetic beads bound with nucleic acids according to the optimized experimental conditions described above (FIG. 2F). The total time required for magnetofluidic-based nucleic acid extraction in the LIAMT device is estimated to be ˜2 min, which is ˜8 times faster than that of the conventional spin-column-based nucleic acid extraction (i.e., ˜15 min) (FIG. 3D).

After the magnetic beads are transferred into the RT-RPA/wax/CRISPR reaction zone, the LIAMT device is inserted into the incubation/detection well for isothermal amplification and CRISPR detection (FIG. 3E). The nucleic acids bound on the magnetic beads release into the RT-RPA reaction mixture and directly serve as the template of the RPA amplification reaction when the processor elevates the temperature to ˜35° C. At 35° C., n-Eicosane wax maintains a solid state because its melting point is typically 36-38° C. After a 30-min pre-amplification, the temperature of the processor is raised to above 40° C., which melts the n-Eicosane wax. With the melting of this wax layer, the RPA amplification product is combined with the CRISPR reaction solution, resulting in specific activation of the CRISPR-Cas12a enzymes in the presence of nucleic acid target. Thus, the activated Cas12a enzyme non-specifically cleaves the cyanine-5 dye (Cy5)/quencher-labeled single-stranded DNA fluorescence probe (ssDNA-Cy5/quencher), generating a strong fluorescence signal for visual detection (see inset of FIG. 3E).

### Detection of Viral RNA by the LIAMT Platform.

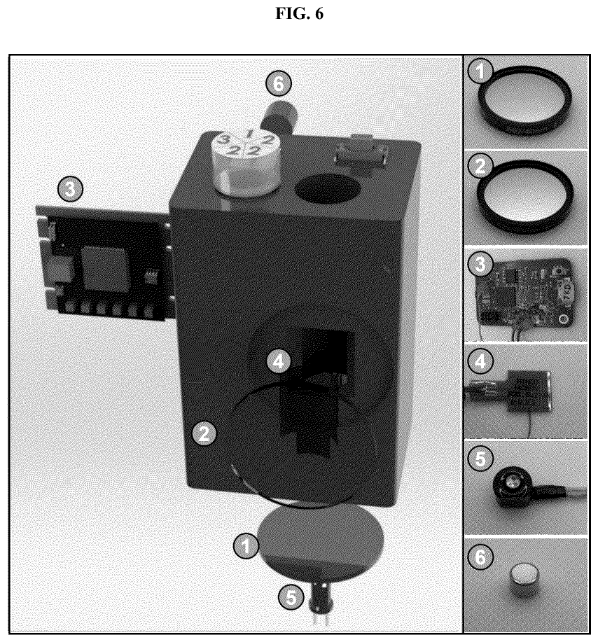

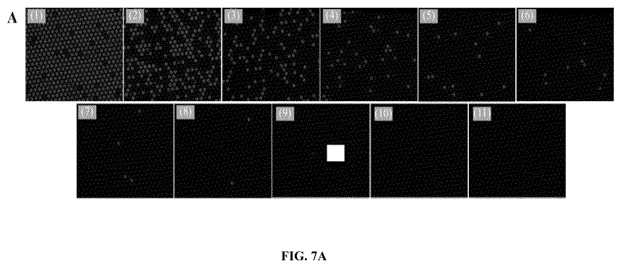

To enable point-of-care diagnostic applications, a portable, palm-sized processor (40 mm×30 mm×60 mm) (FIGS. 3C, 3E, and 6) matched with the disposable LIAMT device was fabricated, eliminating the need for expensive equipment. The processor can be powered by a portable charger. The palm-sized processor features three major functions: i) facilitating magnetic bead separation and transferring by magnetofluidic operation, ii) incubating the LIAMT device at desired temperatures for the RT-RPA reaction and CRISPR cleavage, and iii) enabling visual endpoint fluorescence detection (FIG. 6). The palm-sized processor contains two functional wells: i) a magnetic separation well containing a neodymium magnet (as indicated by the arrow in the processor) (FIG. 3C), designed for the magnetofluidic operation and magnetic bead separation, and ii) an incubation/detection well, for RT-RPA/CRISPR reaction incubation and visual fluorescence detection. To incubate the LIAMT device for RT-RPA/CRISPR, a thin-film heater is attached to the wall of the incubation/detection well (FIG. 6). To observe the endpoint fluorescence, a 645-nm LED was used as the excitation light source and Cy5 fluorescence filter set for fluorescence detection (FIG. 6).

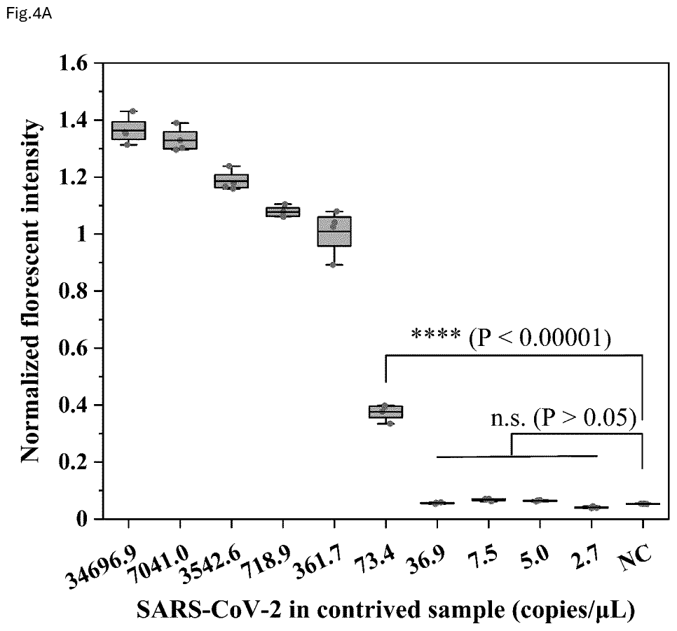

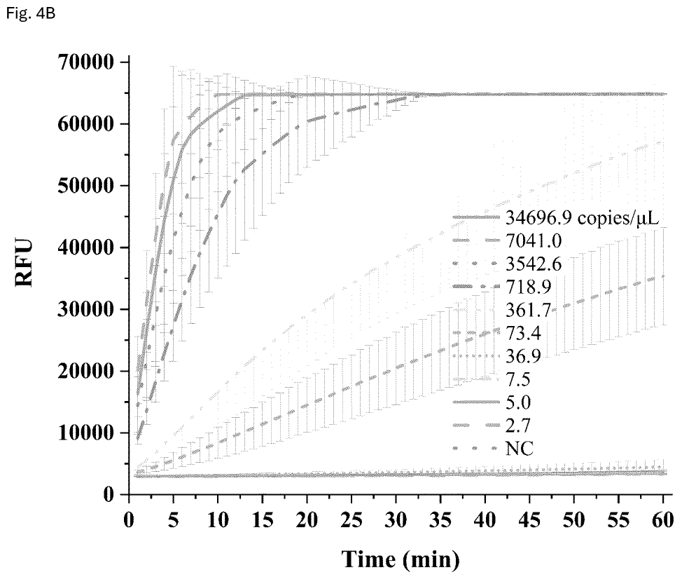

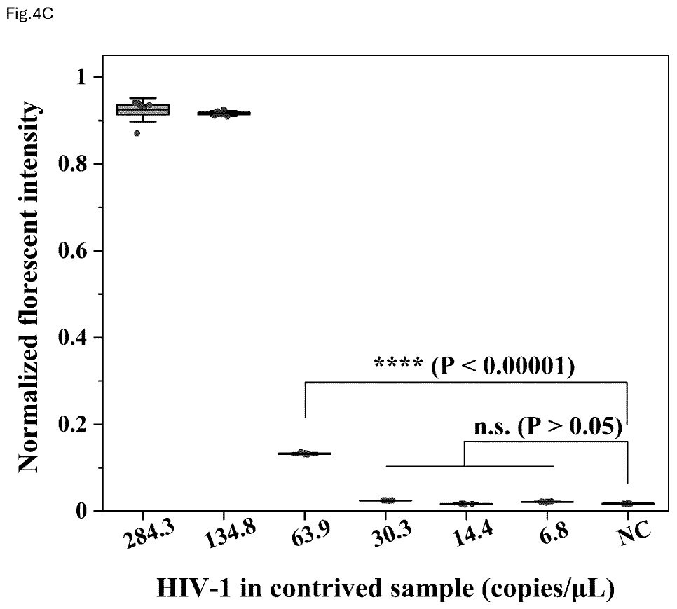

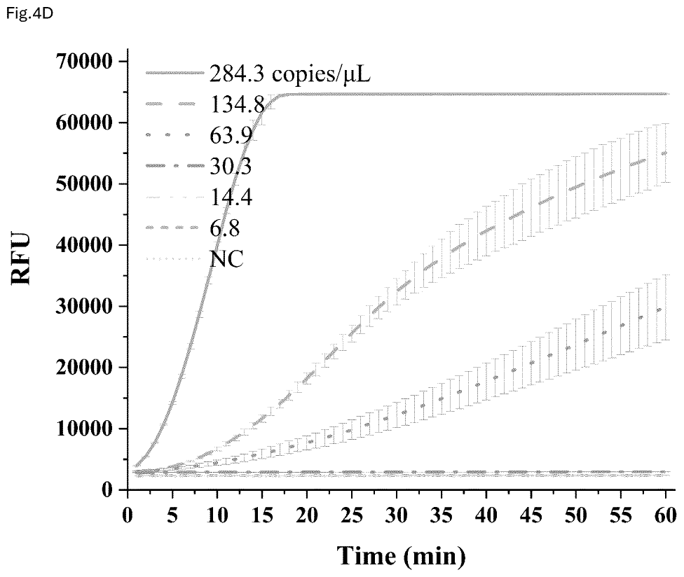

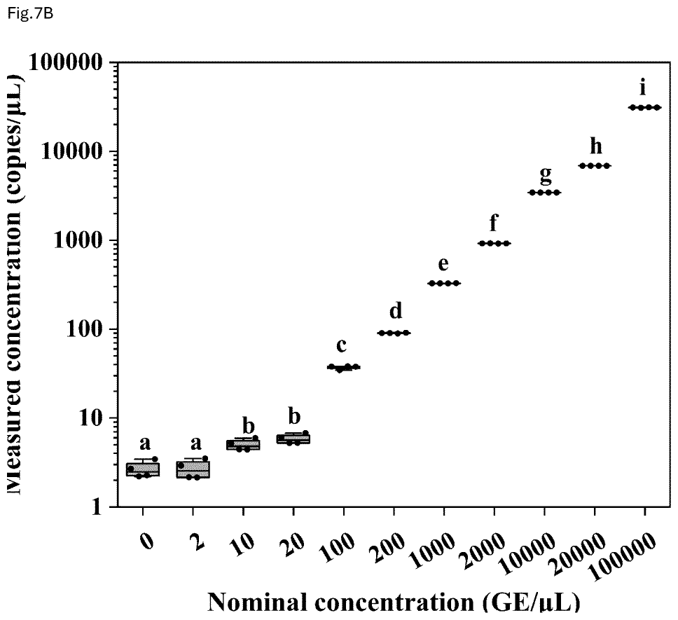

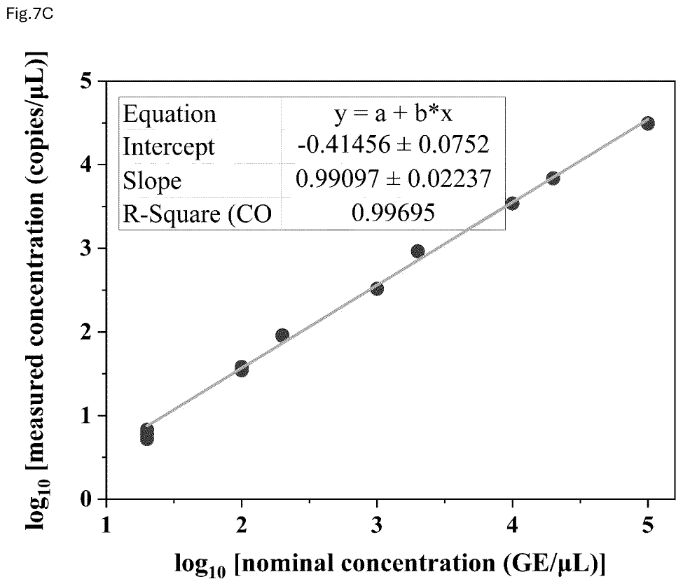

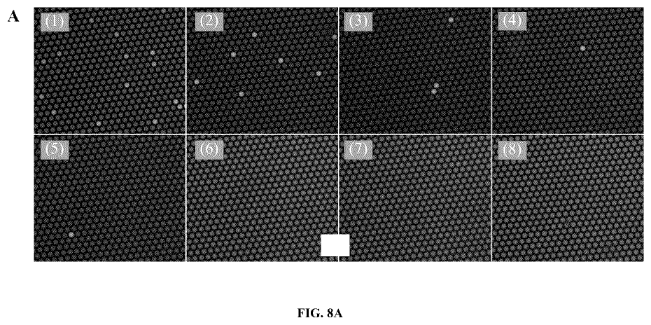

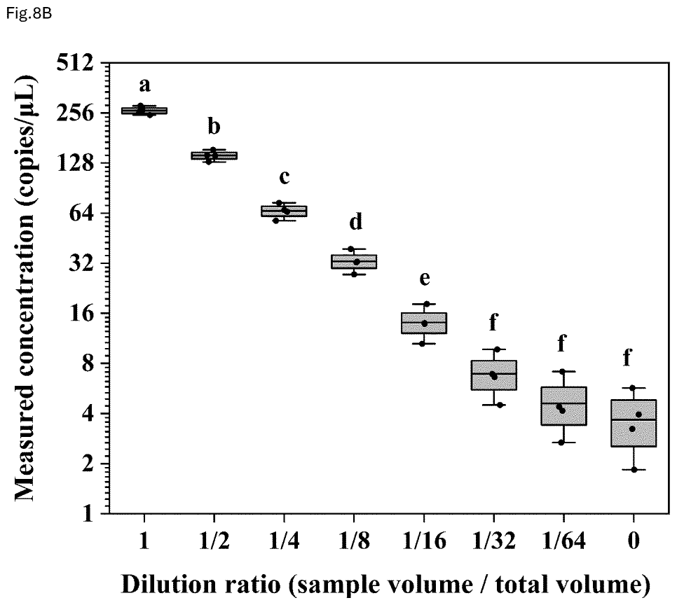

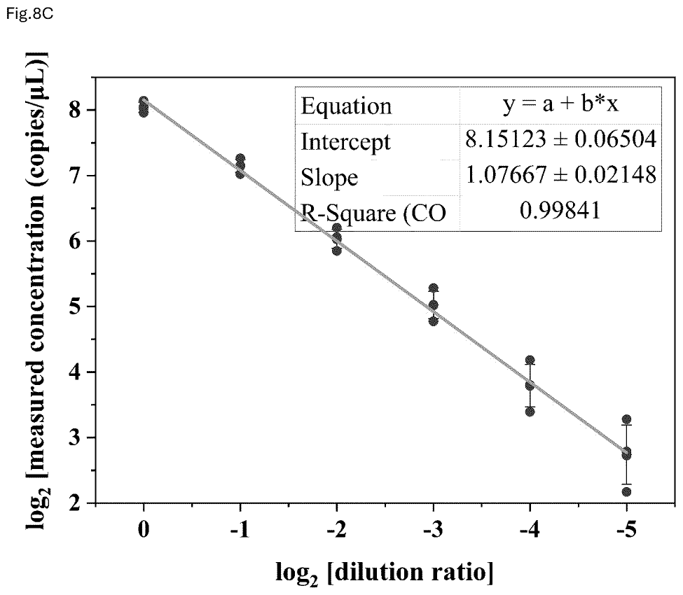

The detection sensitivity of the LIAMT device was evaluated using serial dilutions of virus in the spiked samples. To enable accurate quantitative evaluation, the copy number of SARS-CoV-2 and HIV in the samples was detected and quantitated by QuantStudio 3D digital PCR chips (FIGS. 7 and 8, respectively). Then, serial dilutions of virus in spiked samples was tested using the LIAMT device. As shown in FIG. 4A, the higher the SARS-CoV-2 concentration, the stronger the observed endpoint fluorescence signal. With the LIAMT platform, it was possible to consistently detect 73.4 copies/μL of SARS-CoV-2 RNA, which is comparable to conventional RT-RPA/CRISPR detection using real-time PCR (FIG. 4B). Additionally, HIV was detected in plasma samples at a sensitivity of 63.9 copies/μL (FIG. 4C), which is consistent with that of the RT-RPA/CRISPR assay (FIG. 4D). Thus, the LIAMT platform provides a simple, reliable, and sensitive approach for virus detection at the point of care without the need for complex equipment.

### Clinical Validation of LIAMT Assay Platform.

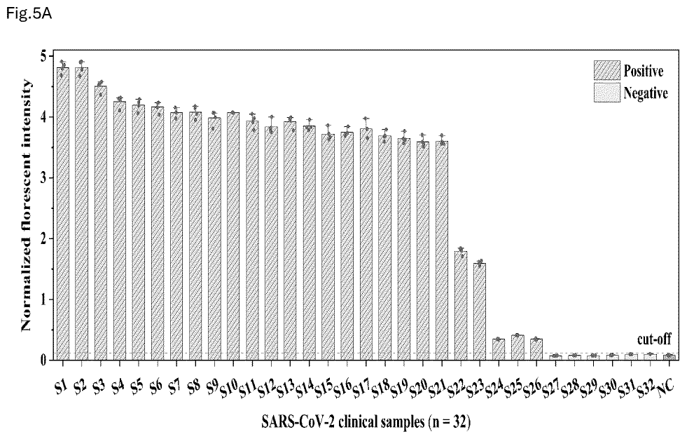

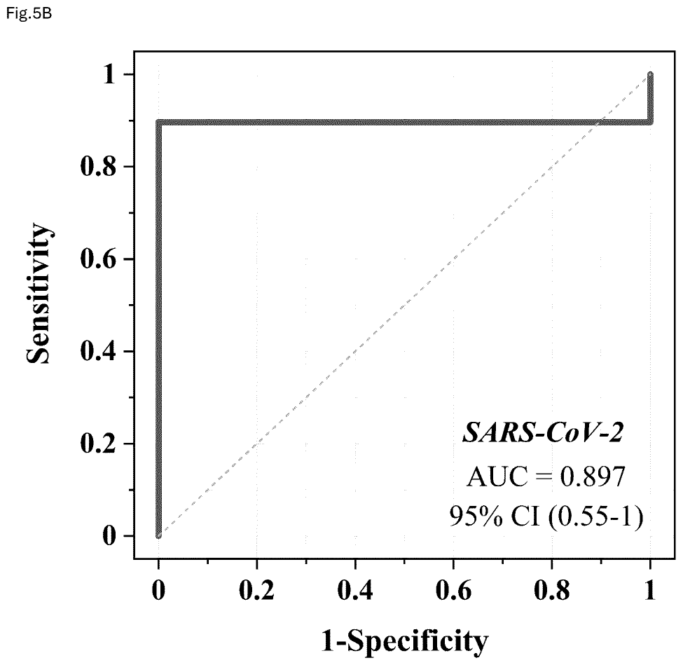

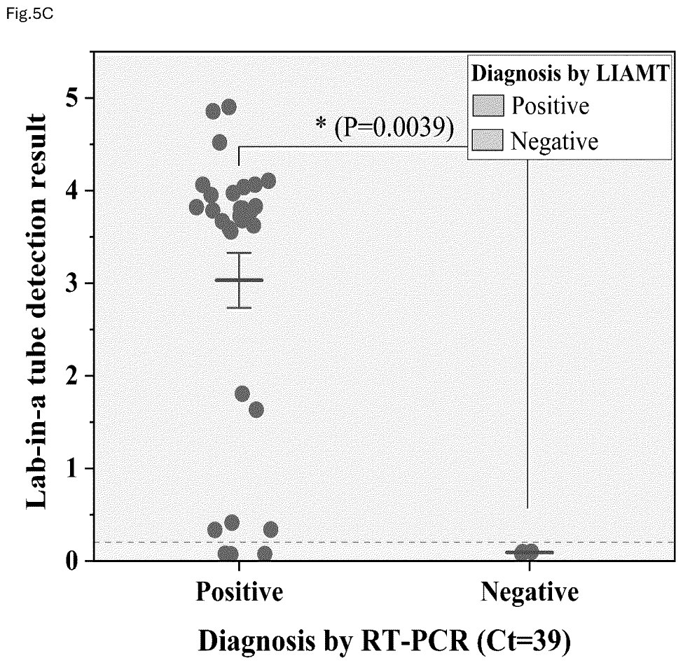

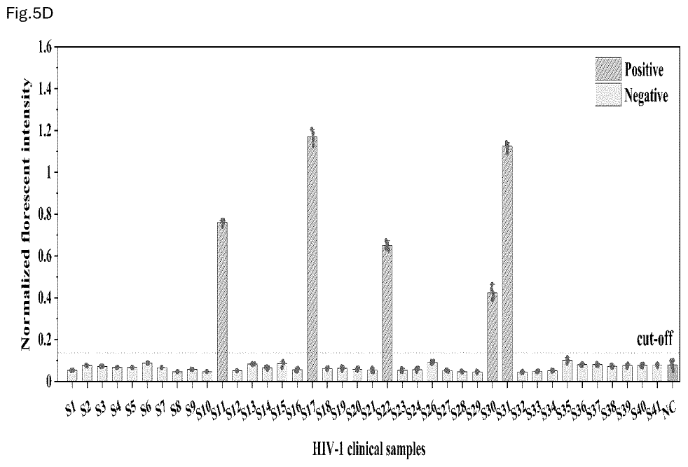

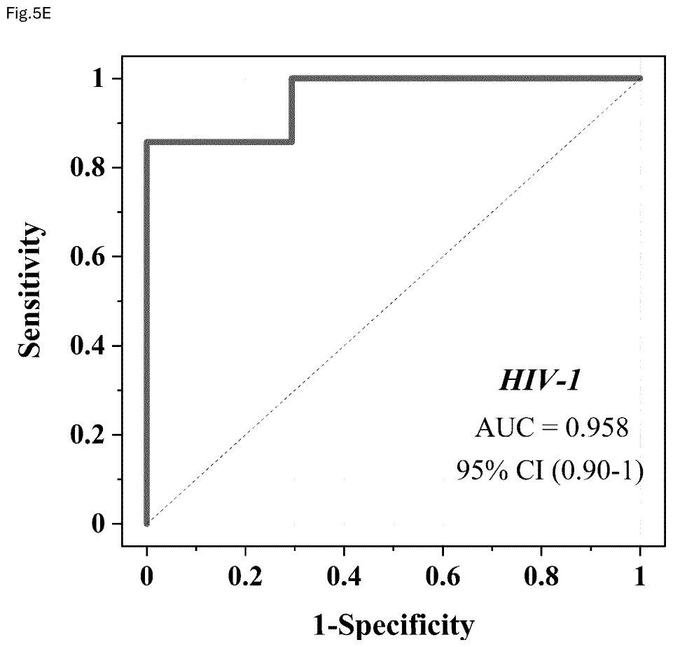

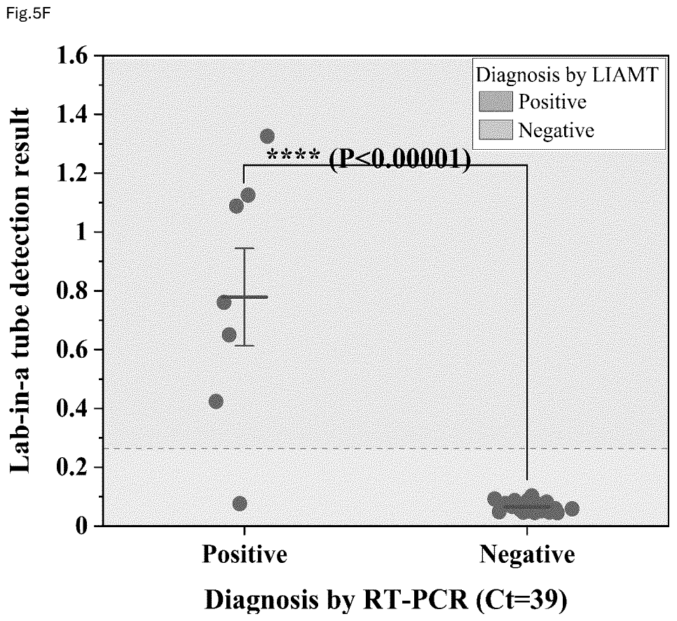

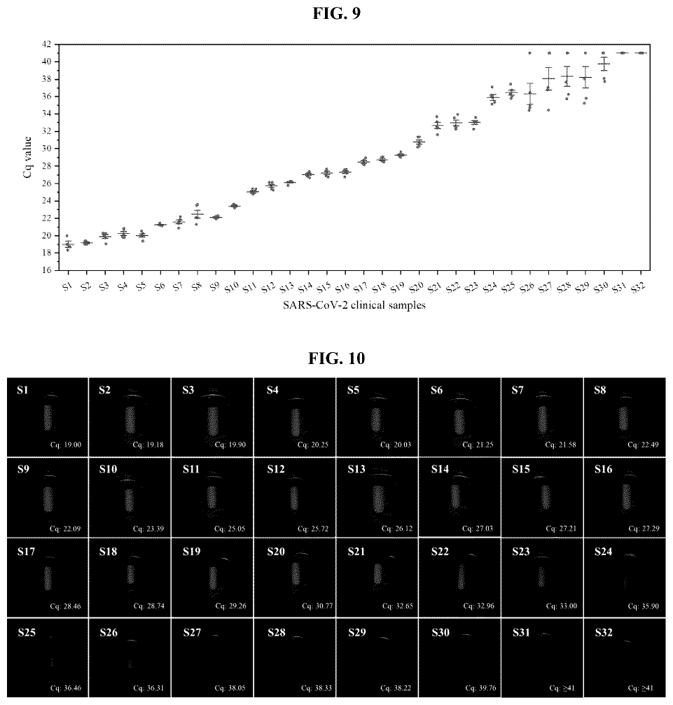

To further validate the clinical utility of the LIAMT platform, a feasibility study was conducted with 73 clinical samples, including 32 clinical nasopharyngeal swab samples for SARS-CoV-2 and 41 clinical plasma samples for HIV. Before testing these samples with the LIAMT platform, RT-PCR was performed to confirm the clinical samples. In clinical diagnostics by PCR/RT-PCR (1), the accepted cut-off for the quantification cycle (Cq) value ranges from 35-40. Typically, samples with Cq>35 are considered to have a low viral load. Here, a cutoff Cq value of 39 was used to determine the sample positivity. As shown in FIG. 9, among the 32 clinical samples, 29 samples (S1-S29) were determined to be positive, and three samples (S30-S32) were determined to be negative. These clinical samples were tested using the LIAMT platform, capturing end-point fluorescence images by a smartphone (FIG. 10). To further analyze the fluorescence images, ImageJ software was used to quantify the fluorescence intensity of the images (FIG. 5A). The fluorescence intensity was normalized and the cut-off threshold set to 0.12, which was calculated by μ±3σ, where μ is the mean value of the negative control and σ is the standard deviation. It was found that lower Cq values of the RT-PCR assay in the clinical samples resulted in stronger fluorescence intensities in the device (FIG. 5A). With the LIAMT platform, 26 samples were distinguished as positive from all 32 clinical samples, consistent with that of the conventional RT-RPA/CRISPR detection (FIG. 11). However, both the LIAMT platform and conventional RT-RPA/CRISPR assay failed to detect three samples (S27, S28, and S29, with Cq values of 38.05, 38.33, and 38.22, respectively) as positive. In a previous study (1), samples with Cq values close to 39 were either weak or false positives. Finally, the receiver operating characteristic (ROC) of the LIAMT platform was assessed using statistical analysis. The LIAMT platform obtained an area under the curve (AUC) of 0.897 (95% confidence interval [CI], 0.55-1) for SARS-CoV-2 detection in clinical samples (FIG. 5B). Additionally, the positive samples showed an elevated fluorescence signal compared to the negative samples (FIG. 5C).

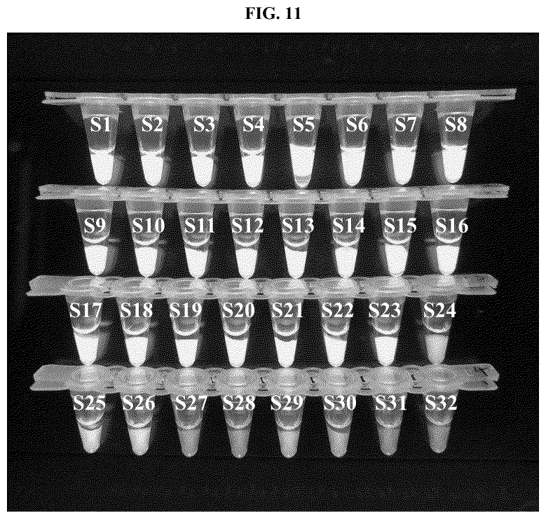

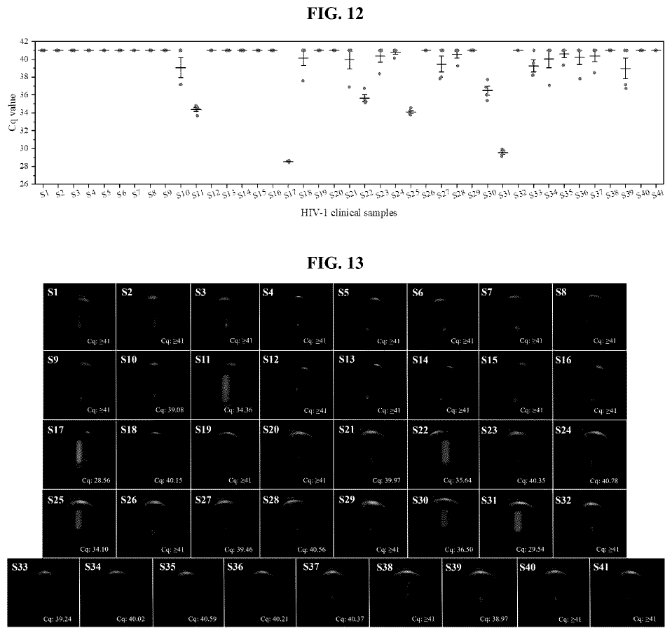

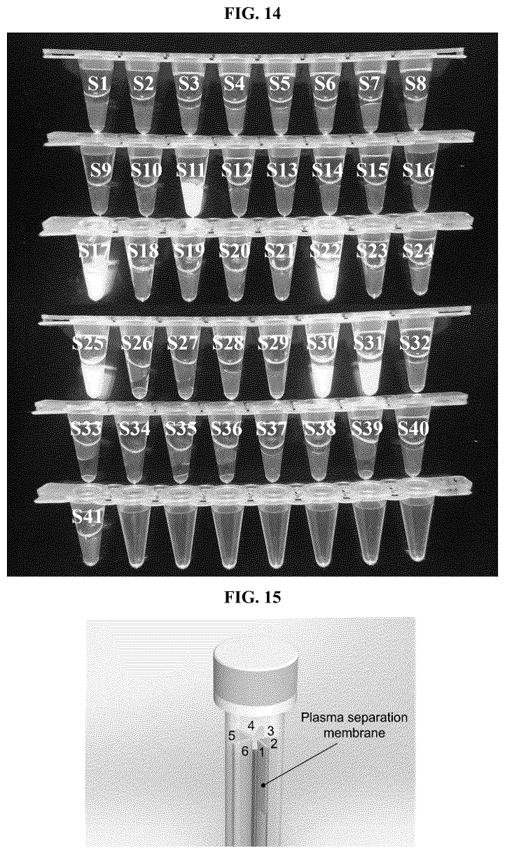

To further evaluate the versatility of the developed LIAMT platform, 41 clinical plasma samples were tested for HIV. Of the 41 clinical samples, seven were determined by RT-PCR as positive for HIV and 34 as negative (FIG. 12). Detecting these clinical samples by the LIAMT platform could distinguish all six positive samples except for sample 39 (FIGS. 5D and 13), consistent with that of the two-step RT-RPA/CRISPR method (FIG. 14). Sample 39 has a Cq value of 38.97, which is close to the cutoff Cq value of 39. To evaluate the assay performance, ROC curve analysis and statistical analysis were performed on the LIAMT clinical results (FIGS. 5E and F). The LIAMT platform showed excellent performance (AUC=0.958, 95% CI, 0.90-1). Therefore, the simple, affordable, and portable LIAMT platform described herein provides a diagnostic platform for virus detection as a versatile diagnostic tool at the point of care.

### Materials and Methods

### Reagents Preparation of LIAMT Assay.

The reagents of the LIAMT assay include lysis/binding buffer, washing buffer, RT-RPA reaction solution, and CRISPR Cas12a reaction mixture. The lysis/binding buffer (D7020-1-100) was obtained from the Quick-DNA/RNA Viral MagBead extraction kit (R2140-E, ZYMO Research, USA). The washing buffer is a 50-mM MgOAc (CAS: 16674-78-5, Sigma-Aldrich) solution with 1% Triton X-100 (CAS: 9036-19-5, Sigma-Aldrich). The RPA kit, TwistAmp® Basic, was obtained from Twist Dx Limited (Maidenhead, UK). RNase H and nuclease-free water were purchased from New England BioLabs (MA, USA). SuperScript™ IV Reverse Transcriptase was purchased from ThermoFisher Scientific (MA, USA). EnGen® Lba Cas 12a was purchased from New England BioLabs (MA, USA). CRISPR RNA (crRNA) and fluorescent reporter (ssDNA-Cy5/quencher) were synthesized from Integrated DNA Technologies (Tables 1 and 2).

TABLE 1
List of all oligonucleotide sequences used for SARS-CoV-2 detection.

| | | SEQ ID |
|---|---|---|
| Oligonucleotide | Sequence | NO |
| crRNA | | |
| LbCas12a-crRNA- | UAAUUUCUACUAAGUGUAGAUUUGAACUGUUGCGACUACGU | 1 |
| SARS-CoV-2 | | |
| Target | | |
| SARS-CoV-2 RNA | from Twist Bioscience Part#102024 (GenBank- | |
| Control 2 | MN908947.3) | |
| RPA Primers | | |
| FP_SARS-CoV-2 | CGGCAGTCAAGCCTCTTCTCGTTCCTCATC | 2 |
| RP_SARS-CoV-2 | CAGACATTTTGCTCTCAAGCTGGTTCAATC | 3 |
| Reporter | | |
| ssDNA-Cy5Q | /5Cy5/TTATTTTATT/3BHQ_2/ | 4 |
| ssDNA-FQ | /56-FAM/TTATT/3IABkFQ/ | 5 |
| PCR | | |
| SARS-CoV-2 Forward | from IDT Part#10006821 | |
| Primer | | |
| SARS-CoV-2 Reverse | from IDT Part#10006822 | |
| Primer | | |
| SARS-CoV-2 PCR | from IDT Part#10006823 | |
| Probe | | |

TABLE 2
List of all oligonucleotide sequences used for HIV detection.

| | | SEQ ID |
|---|---|---|
| Oligonucleotide | Sequence | NO |
| crRNA | | |
| Lba Cas12a-crRNA1- | UAAUUUCUACUAAGUGUAGAUAUCCCAUUCUGCAGCUUCCU | 6 |
| HIV-1 | CAUU | |
| Lba Cas12a-crRNA2- | UAAUUUCUACUAAGUGUAGAUUUGCACCAGGCCAGAUAAGA | 7 |
| HIV-1 | | |
| Target | | |
| HIV-1 plasmid | CCAGAAGTAATACCCATGTTTTCAGCATTATCAGAAGGAGCCA | 8 |
| | CCCCACAAGATTTAAACACCATGCTAAACACAGTGGGGGGACA | |
| | TCAAGCAGCCATGCAAATGTTAAAAGAAACCATCAATGAGGAA | |
| | GCTGCAGAATGGGATGAAATGCATCCCGTGCAGGCAGGGTTTG | |
| | TTGCACCAGGCCAGATAAGAGATCCAAGGGGAAGTGACATAGC | |
| | AGGAACTACCAGTACCCTTCAGGAACAAATAGGATGGATGACA | |
| | AGTAATCCACCTATCCCAGTAGGAGAAATCTATAAAAGATGG | |
| AcroMetrix HIV-1 high | from ThermoFisher Scientific Cat. #964003 | |
| controls | | |
| ACCURUN 315 series | from SeraCare Life Sciences Cat. #2020-0092 | |
| 400 HIV-1 RNA positive | | |
| control | | |
| RPA Primers | | |
| FP_HIV-1 | CAAGCAGCCATGCAAATGTTAAAAGAAACCATC | 9 |
| RP_HIV-1 | GTAGTTCCTGCTATGTCACTTCCCCTTGGATC | 10 |
| dsDNA | | |
| RPA amplicons | CAAGCAGCCATGCAAATGTTAAAAGAAACCATCAATGAGGAAG | 11 |
| | CTGCAGAATGGGATGAAATGCATCCCGTGCAGGCAGGGTTTGT | |
| | TGCACCAGGCCAGATAAGAGATCCAAGGGGAAGTGACATAGC | |
| | AGGAACTAC | |
| Reporter | | |
| MB-Invertase reporter | MB/streptavidin/BsI probe | |
| BsI probe | /5Biotin/AAAAAAAAAAAATCAATCTCGGATTCCGGAGACAAGTT | 12 |
| | GAAGAGAACCTGGGGGAGTGCGAGTTCACAGATGAGTTGACAG | |
| | ATGAGTAAAAAAAAAAAA/3Invertase | |
| ssDNA-FQ | /56-FAM/TTATT/3IABkFQ/ | 13 |
| PCR | | |
| HIV-1 Forward Primer | CAAGCAGCCATGCAAATGTTA | 14 |
| HIV-1 Reverse Primer | GGTAGTTCCTGCTATGTCACTTC | 15 |
| HIV-1 Probe | /56-FAM/TTGGATCTC/ZEN/TTATCTGGCCTGGTGC/3IABkFQ/ | 16 |

The RT-RPA/wax/CRISPR reaction zone contains 50 μL of RT-RPA reaction mixture, 20 μL of n-Eicosane wax, and 60 μL of CRISPR Cas12a reaction solution. A total of 60 μL of CRISPR Cas12a reaction mixture was prepared, containing 400 nM Lba Cas12a, 400 nM crRNA, 1×NEBuffer 2.1 (50 mM NaCl, 10 mM Tris-HCl, 10 mM MgCl₂, 100 ag/mLh BSA, pH 7.9, 25′C), and 4 μM ssDNA-Cy5/quencher fluorescent reporter. The prepared CRISPR mixture was pre-stored in the device and sealed with 20 μL of n-Eicosane wax (CAS: 112-95-8, Sigma-Aldrich). The RT-RPA reaction mixture (in 50 μL) contained one dried pellet, 29.5 μL of primer-free rehydration buffer, 0.5 μM each for forward and reverse primers, 2 U/μL Superscript IV reverse transcriptase, 0.1 U/μL RNase H, and 14 mM MgOAc. In the LIAMT assay, RT-RPA reaction mixture was directly added on the top of the wax and formed an RT-RPA/wax/CRISPR reaction system.

### LIAMT Device Design and Fabrication.

The LIAMT device includes a 3D-printed separator and a microcentrifuge tube (1.5-mL conical screw cap tubes, 02-681-373, Fisher Scientific, NH, USA). The separator was designed using Solidworks software and fabricated by a Form 3 3D printer using clear resin (RS-F2-GPCL-04, Formlabs, MA, USA). The separator divided the microcentrifuge tube into five independent zones, including a lysis/binding buffer zone (with a 90-degree angle), three wash zones (with a 70-degree angle each), and an RT-RPA/wax/CRISPR detection zone (with a 60-degree angle) (as shown in the insets of FIGS. 1B and 3A). Next, the 3D-printed separator was washed with isopropyl alcohol for at least 30 min and cured under ultraviolet (UV) light for at least 3 hours. After UV curing, the edges of the separator were coated with polydimethylsiloxane (PDMS) solution (RTV615 001-KIT, Momentive Performance Materials, NY, USA). Then, the 3D-printed separator coated with PDMS solution was inserted into a microcentrifuge tube. The sealing with PDMS ensures no liquid leakage between different buffer zones. Last, the assembled LIAMT devices were placed at room temperature for more than 12 hours, allowing the PDMS to completely solidify.

### Portable Palm-Sized Processor Development.

The palm-sized processor was designed using Solidworks and fabricated by a Form 3 3D printer using black resin (RS-F2-GPBK-04, Formlabs, MA, USA). The processor has two vial wells: i) one magnetofluidic separation well for magnetofluidic transferring and operation, and ii) one incubation/detection well for RT-RPA/CRISPR incubation and fluorescence detection (FIG. 3C). In the magnetofluidic separation well, a cylindrical neodymium magnet (6.33 mm diameter×6.33 mm height) was embedded to separate the magnetic beads and transfer them into different functional zones of the device through magnetic force. In the incubation/detection well, a flexible thin-film heater (HK5572R26.5L23A, Minco, MN, USA) and a thermocouple wire were fixed on its inner wall to heat the device and monitor the temperature, respectively (FIG. 6).

To visualize the fluorescence signal of the LIAMT device, a Cy5 fluorescence filter set (#67-010, Edmund Optics, NJ, USA) along with LED lights (LED645L, Thorlabs, NJ, USA) were used to build an optical detection path. The LED was placed at the bottom of the incubation/detection well and served as an excitation light source (FIG. 6). To block light with other wavelengths, the excitation filter (#67-035 in Cy5 fluorescence filter set) was used to cover the LED. To obtain clear fluorescence signal from the LIAMT device, the emission filter (#67-038 in the Cy5 fluorescence filter set) was placed in the observation window of the processor (FIG. 6).

### Operation and Detection of LIAMT Platform.

For clinical testing with the LIAMT device, 60-μL clinical samples were added into the lysis/binding buffer zone containing pre-stored lysis/binding buffer (120 μL) and magnetic beads (2 μL) (Quick-DNA/RNA Viral MagBead extraction kit). After samples were introduced and mixed with the lysis/binding buffer, mineral oil (#1632129, Bio-Rad, USA) was added into the LIAMT device to cover all buffer solutions, thereby completely isolating the individual zones. Then, the device was sealed by a screw cap marked by yellow tape, indicating the location of the different zones (FIGS. 3B and C). The numbers 1, 2, and 3 on the screw cap show the lysis/binding buffer zone, the washing buffer zone, and the RT-RPA/wax/CRISPR reaction zone, respectively (FIGS. 3B and C). The location of the neodymium magnet was marked with an arrow in the magnetofluidic separation well of the processor (FIGS. 3B and C). After the device was slowly inserted into the magnetofluidic separation well by aligning its lysis/binding zone (as indicated by “1” on the cap) with the magnet (as indicated by the arrow in the processor), the magnetic beads were collected and moved into the mineral oil layer by magnetic force. Then, the LIAMT device was slowly rotated clockwise to the first washing buffer zone (as indicated by “2” on the cap) and was manually lifted, which brought the magnetic beads into the first washing buffer for purification. After two more washing steps by the same operations, the magnetic beads bound with nucleic acids were transferred into the RT-RPA/wax/CRISPR reaction zone. Last, the LIAMT device was inserted into the incubation/detection well of the palm-sized processor for the RT-RPA reaction and CRISPR detection. The thin-film flexible heater first heated the device for the RPA pre-amplification reaction at ˜35° C., in which the n-Eicosane wax remains solid and isolates the RT-RPA reaction mixture and CRISPR reaction solution. After a 30-min RPA preamplification, the heater's temperature was elevated to ˜40° C. The elevated temperature resulted in the melting of the n-Eicosane wax, which brought the RPA pre-amplification reaction mixture and CRISPR reaction solution together and initiated the CRISPR detection. After a 30-min CRISPR reaction, the fluorescence signal of the CRISPR reaction was visually observed by the naked eye through the observation window on the processor (FIG. 3E).

### Selection and Optimization of Lysis/Binding Buffer and Washing Buffer.

Five lysis/binding buffer solutions were compared and tested, including: i) ZYMO lysis/binding buffer (R2140-E, ZYMO Research, USA), ii) MES binding buffer (4 M guanidine thiocyanate (GuSCN), 10 mM MES [2-ethanesulfonic acid], 1% Triton X-100, 1% β-mercaptoethanol), iii) MgOAc lysis/binding buffer (5 M guanidine thiocyanate, 100 mM MgOAc, 1% Triton X-100), iv) Tris-HCl lysis/binding buffer (4 M guanidine thiocyanate, 55 mM Tris HCl pH 7.5, 25 mM EDTA, and 3% Triton X-100), and v) Tris-HCl with isopropanol binding buffer (50% v/v Tris-HCl binding buffer and 50% v/v isopropanol). In addition, four washing buffer solutions were prepared and tested, including: i) deionized water, ii) 70% ethanol, iii) MgOAc with 1% Triton X-100, and iv) 20% PEG solution. For the optimization of MgOAc washing buffer, six MgOAc washing buffer solutions with various concentrations (1, 5, 10, 50, 100, and 500 mM with 1% Triton X-100) were prepared and tested. To optimize the usage of the magnetic beads and washing buffer, serial volumes of magnetic beads (0.5, 1, 2, 4, 8, and 16 μL) and washing buffer (120, 180, 240, 300, 360, and 420 μL) were prepared and tested. Additionally, different washing operations (e.g., 1, 2, 3, and 4 washing steps) were evaluated and compared in the LIAMT device.

### Preparation and Detection of Spiked Samples and Clinical Samples.

Before detection on clinical samples, swab samples spiked with heat-inactivated SARS-CoV-2 virus (Isolate USA-WA1/2020, BEI, USA) were used to evaluate the analytical performance of the LIAMT device. For HIV detection, AcroMetrix™ HIV-1 High Control (Thermo Fisher) was used to prepare spiked plasma samples. Before testing, the copy numbers of the SARS-CoV-2 and HIV in the samples were quantified by digital RT-PCR. De-identified nasopharyngeal swab samples for SARS-CoV-2 and clinical plasma samples for HIV were prepared and tested under the approval of the Institutional Review Board of the University of Connecticut Health Center (protocol #P61067). For RT-PCR or RT-RPA/CRISPR detection, the viral RNA samples were extracted using the QIAamp Viral RNA Mini Kit (Qiagen) according to the manufacturer's protocol.

### Rt-PCR and Rt-RPA/CRISPR Detection.

A real-time fluorescence RT-PCR assay was used to detect the extracted RNA as the gold-standard method. The GoTaq Probe 1-Step RT-qPCR kit from Promega (WI, USA) was used to prepare the RT-PCR reaction solution. A total 20 μL of RT-PCR mix was used, including 1×GoTaq Probe Master Mix, 1×GoScript RT Mix for 1-Step RT-qPCR, 0.5 M forward primer, 0.5 M reverse primer, 0.2 μM probe, and 2 μL of the target. The RT-PCR protocol contains three steps: i) reverse transcription (15 min at 45° C.), ii) RT inactivation and polymerase activation (2 min at 95° C.), and iii) denaturation and extension (40 cycles of 15 s at 95° C. and 60 s at 60° C.). Real-time RT-PCR detection was performed using the Bio-Rad CFX96 Touch Real-Time PCR Detection System.

For comparison purpose with the LIAMT device, the conventional RT-RPA/CRISPR was applied to test the extracted RNA in the samples. For the RT-RPA reaction, the extracted RNA from the samples was added to the RT-RPA reaction mixture. After the RT-RPA reaction, 2 μL of RT-RPA amplification product was introduced to the CRISPR reaction mixture for CRISPR detection. ssDNA-FQ probe was used for the real-time CRISPR fluorescence detection by a real-time PCR machine.

### Digital PCR Quantification for Target.

BioRad Reliance One-Step Multiplex RT-qPCR Supermix (Cat. #12010176) was purchased from BioRad. The RT-PCR mixture was prepared according to the manufacturer's protocol. After 14.5 μL of reaction mixture was loaded into the 3D Digital PCR chip (Cat. #A26316) by using a 3D Digital PCR Chip Loader (Cat. #A29154), the chip was placed on a ProFlex 2× Flat Block Thermal Cycler (Cat. #A26316) for the PCR reaction. At the end of the RT-PCR reaction, the chip was read on a QuantStudio 3D Digital PCR Instrument (Cat. #A26316) or a fluorescence microscope Axio Observer from ZEISS.

### Statistical Analysis.

Statistical analyses were performed by OriginLab 2020. All statistical analyses were performed using multiple Student's T-test, where n.s.=not significant with p>0.05, and asterisks (*, **, ***, ****) denoting significant differences with the following p values (*=0.001<p≤0.05, **=0.0001<p≤0.001, ***=0.00001<p≤0.0001, ****=p≤0.00001).

### Example 2

A Lab-in-a Magnetofluidic Tube (LIAMT) incorporated with plasma separation membrane was developed. FIG. 15 shows a schematic illustration of the fully integrated and self-contained LIAMT device incorporated with Vivid plasma separation membrane, which is capable of performing plasma separation, nucleic acid sample preparation, isothermal amplification, CRISPR cleavage reaction, and fluorescence detection in a single engineered tube. The disposable LIAMT device is composed of a 3D-printed separator and a commercially available 1.5 mL microcentrifuge tube. The separator isolates the microcentrifuge tube into six independent zones: i) one blood sample zone with Vivid plasma separation membrane for plasma separation (zone 1), ii) one lysis/binding buffer zone for viral lysis and nucleic acid binding on the magnetic beads (zone 2), iii) three washing buffer zones for nucleic acid purification (zones 3-5), and iv) one RT-RPA/wax/CRISPR reaction zone (zone 6). All buffer solutions and reagents are pre-stored in the LIAMT device and covered by mineral oil.

In addition, a portable Raspberry Pi Processor for Automatic Magnetofluidic Operation and Detection of the LIAMT Device was developed. FIG. 16 shows a schematic illustration of the portable Raspberry Pi processor and single well device, which is powered by a Raspberry Pi 4B. It can automatically perform magnetofluidic operation for nucleic acid sample preparation, isothermal amplification, CRISPR cleavage reaction, fluorescence signal detection and nucleic acid quantification. When testing clinical samples, the sample is first introduced into the LIAMT device. Then, the device is inserted into the Raspberry Pi processor (FIG. 16A). The RasPi brain controls an Adafruit motor driver with two stepper motors to move the magnetic beads in the LIAMT device for nucleic acid sample preparation and purification. Next, the RasPi activates the heating module to provide elevated temperature for the RPA and CRISPR reactions. During the CRISPR reaction, the processor will real-time monitor the fluorescence signals, analyze the fluorescence images and quantify the nucleic acid target, enabling an automatic, “sample-to-result” quantitative detection of pathogens in clinical samples (FIG. 16B).

For reasons of completeness, various aspects of the disclosure are set out in the following numbered clauses:

Clause 1. A device, comprising:
a reaction vessel comprising a plurality of distinct assay zones comprising a) a lysis zone comprising a lysis/binding buffer and magnetic beads; b) one or more washing zones comprising a washing buffer; c) an amplification zone comprising amplification reagents; and d) a detection zone comprising nucleic acid detection reagents.

Clause 2. The device of clause 1, wherein said assay zones are vertically oriented columns in the reaction vessel.

Clause 3. The device of clause 2, wherein the assay zones are separated by a separator.

Clause 4. The device of any of clauses 1-3, wherein the top of the reaction vessel is covered with mineral oil in fluid contact with the top surface of each of the assay zones.

Clause 5. The device of any of clauses 1-4, wherein the amplification zone and the detection zone are in the same assay zone and are separated by wax.

Clause 6. The device of any of clauses 1-5, wherein the device further comprises a blood sample zone comprising a plasma separation membrane.

Clause 7. The device of any of clauses 1-6, wherein the one or more washing zones are three washing zones.

Clause 8. The device of any of clauses 1-7, wherein the amplification reagents are reagents for isothermal amplification of a target nucleic acid.

Clause 9. The device of clause 8, wherein the isothermal amplification is recombinase polymerase amplification (RPA) or reverse transcriptase RPA (RT-RPA).

Clause 10. The device of any of clauses 1-9, wherein the nucleic acid detection reagents are reagents for performing a CRISPR assay.

Clause 11. The device of any of clauses 1-10, wherein the reaction chamber is a reaction tube.

Clause 12. The device of any of clauses 1-11, wherein the device is disposable.

Clause 13. A system, comprising a) the device of any of clauses 1-12; and b) a sample processor comprising a magnetofluidic separation component for magnetofluidic transferring and operation and an incubation/detection component.

Clause 14. The system of clause 13, wherein the magnetofluidic separation component and the incubation/detection component comprise a magnetofluidic separation well and an incubation/detection well or a single well configured for magnetofluidic separation and incubation/detection.

Clause 15. The system of clause 13, wherein the magnetofluidic well comprises a magnet and/or the incubation/detection well comprises a heater and a thermocouple wire.

Clause 16. The system of any of clauses 13-15, further comprising one or more additional components selected from a microprocessor, a plurality of first stepper motors configured to transfer magnetic beads within assay zones of the device, a plurality of second stepper motors configured to move the device between the magnetofluidic well and the incubation/detection well, and a camera.

Clause 17. A method for detection of a target nucleic acid in a sample, comprising:
a) contacting the sample with the system of any of clauses 13-16; and
b) detecting the presence and/or level of the nucleic acid in the sample using the system.

Clause 18. The method of clause 17, wherein the nucleic acid is DNA or RNA.

Clause 19. The method of clause 17 or clause 18, wherein the nucleic acid is a pathogen nucleic acid.

Clause 20. The method of clause 19, wherein the pathogen is a virus selected from SARS-CoV-2 and HIV.

### REFERENCES

1. Z. Li, N. Uno, X. Ding, L. Avery, D. Banach, C. Liu, Bioinspired CRISPR-Mediated Cascade Reaction Biosensor for Molecular Detection of HIV Using a Glucose Meter. ACS Nano 17, 3966-3975 (2023).
2. R. Nouri, Y. Jiang, A. J. Politza, T. Liu, W. H. Greene, Y. Zhu, J. J. Nunez, X. Lian, W. Guan, STAMP-Based Digital CRISPR-Cas13a for Amplification-Free Quantification of HIV-1 Plasma Viral Loads. ACS Nano 17, 10701-10712 (2023).
3. X. Wang, H. Yao, X. Xu, P. Zhang, M. Zhang, J. Shao, Y. Xiao, H. Wang, Limits of Detection of 6 Approved RT-PCR Kits for the Novel SARS-Coronavirus-2 (SARS-CoV-2). Clin Chem 66, 977-979 (2020).
4. N. Lübke, T. Senff, S. Scherger, S. Hauka, M. Andrée, O. Adams, J. Timm, A. Walker, Extraction-free SARS-CoV-2 detection by rapid RT-qPCR universal for all primary respiratory materials. Journal of Clinical Virology 130, 104579 (2020).
5. C. Liu, E. Geva, M. Mauk, X. Qiu, W. R. Abrams, D. Malamud, K. Curtis, S. M. Owen, H. H. Bau, An isothermal amplification reactor with an integrated isolation membrane for point-of-care detection of infectious diseases. Analyst 136, 2069-2076 (2011).
6. J. Wen, L. A. Legendre, J. M. Bienvenue, J. P. Landers, Purification of Nucleic Acids in Microfluidic Devices. Anal Chem 80, 6472-6479 (2008).
7. Y.-K. Cho, J.-G. Lee, J.-M. Park, B.-S. Lee, Y. Lee, C. Ko, One-step pathogen specific DNA extraction from whole blood on a centrifugal microfluidic device. Lab Chip 7, 565-573 (2007).
8. H. T. Ngo, M. Jin, A. Y. Trick, F.-E. Chen, L. Chen, K. Hsieh, T.-H. Wang, Sensitive and Quantitative Point-of-Care HIV Viral Load Quantification from Blood Using a Power-Free Plasma Separation and Portable Magnetofluidic Polymerase Chain Reaction Instrument. Anal Chem 95, 1159-1168 (2023).
9. S. M. Berry, E. T. Alarid, D. J. Beebe, One-step purification of nucleic acid for gene expression analysis via Immiscible Filtration Assisted by Surface Tension (IFAST). Lab Chip 11, 1747-1753 (2011).
10. Z. Huang, C. J. Lyon, T. Y. Hu, CRISPR-based assays for low-resource settings. Nature Reviews Bioengineering 1, 230-231 (2023).
11. X. Ding, K. Yin, Z. Li, R. V. Lalla, E. Ballesteros, M. M. Sfeir, C. Liu, Ultrasensitive and visual detection of SARS-CoV-2 using all-in-one dual CRISPR-Cas12a assay. Nat Commun 11, 1-10 (2020).
12. S. Berensmeier, Magnetic particles for the separation and purification of nucleic acids. Appl Microbiol Biotechnol 73, 495-504 (2006).
13. H. He, R. Li, Y. Chen, P. Pan, W. Tong, X. Dong, Y. Chen, D. Yu, Integrated DNA and RNA extraction using magnetic beads from viral pathogens causing acute respiratory infections. Sci Rep 7, 45199 (2017).
14. Z. Li, Y. Bai, M. You, J. Hu, C. Yao, L. Cao, F. Xu, Fully integrated microfluidic devices for qualitative, quantitative and digital nucleic acids testing at point of care. Biosens Bioelectron 177, 112952 (2021).
15. H. Lin, W. Yu, K. A. Sabet, M. Bogumil, Y. Zhao, J. Hambalek, S. Lin, S. Chandrasekaran, O. Garner, D. Di Carlo, S. Emaminejad, Ferrobotic swarms enable accessible and adaptable automated viral testing. Nature 611, 570-577 (2022).
16. J. Song, C. Liu, M. G. Mauk, J. Peng, T. Schoenfeld, H. H. Bau, A Multifunctional Reactor with Dry-Stored Reagents for Enzymatic Amplification of Nucleic Acids. Anal Chem 90, 1209-1216 (2018).
17. Q. Bao, J. Sun, X. Fu, L. Sheng, Y. Ye, J. Ji, Y. Zhang, J. Wang, J. Ping, X. Sun, A Simplified Amplification-Free Strategy with Lyophilized CRISPR-CcrRNA System for Drug-Resistant Salmonella Detection. Small 19, 2207343 (2023).

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

### Other Embodiments

Embodiments disclosed here are not limiting of the subject matter and is merely exemplary. Various other components may be included and called upon for providing for aspects of the teachings herein. For example, additional materials, combinations of materials and/or omission of materials may be used to provide for added embodiments that are within the scope of the teachings herein.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A device, comprising:
A reaction vessel comprising a plurality of distinct assay zones comprising a) a lysis zone comprising a lysis/binding buffer and magnetic beads; b) one or more washing zones comprising a washing buffer; c) an amplification zone comprising amplification reagents;
and d) a detection zone comprising nucleic acid detection reagents.

2. The device of claim 1, wherein said assay zones are vertically oriented columns in the reaction vessel.

3. The device of claim 2, wherein the assay zones are separated by a separator.

4. The device of claim 1, wherein the top of the reaction vessel is covered with mineral oil in fluid contact with the top surface of each of the assay zones.

5. The device of claim 1, wherein the amplification zone and the detection zone are in the same assay zone and are separated by wax.

6. The device of claim 1, wherein the device further comprises a blood sample zone comprising a plasma separation membrane.

7. The device of claim 1, wherein the one or more washing zones are three washing zones.

8. The device of claim 1, wherein the amplification reagents are reagents for isothermal amplification of a target nucleic acid.

9. The device of claim 8, wherein the isothermal amplification is recombinase polymerase amplification (RPA) or reverse transcriptase RPA (RT-RPA).

10. The device of claim 1, wherein the nucleic acid detection reagents are reagents for performing a CRISPR assay.

11. The device of claim 1, wherein the reaction chamber is a reaction tube.

12. The device of claim 1, wherein the device is disposable.

13. A system, comprising a) the device of claim 1; and b) a sample processor comprising a magnetofluidic separation component for magnetofluidic transferring and operation and a incubation/detection component.

14. The system of claim 13, wherein the magnetofluidic separation component and the incubation/detection component comprise a magnetofluidic separation well and an incubation/detection well or a single well configured for magnetofluidic separation and incubation/detection.

15. The system of claim 14, wherein the magnetofluidic well comprises a magnet.

16. The system of claim 14, wherein the incubation/detection well comprises a heater and a thermocouple wire.

17. The system of claim 13, further comprising one or more additional components selected from a microprocessor, a plurality of first stepper motors configured to transfer magnetic beads within assay zones of the device, a plurality of second stepper motors configured to move the device between the magnetofluidic well and the incubation/detection well, and a camera.

18. A method for detection of a target nucleic acid in a sample, comprising:
a) contacting the sample with the system of claim 13; and
b) detecting the presence and/or level of the nucleic acid in the sample using the system.

19. The method of claim 18, wherein the nucleic acid is DNA or RNA.

20. The method of claim 19, wherein the nucleic acid is a pathogen nucleic acid selected from SARS-CoV-2 and HIV.
